(19) United States
(12) Patent Application Publication
(10) Pub. No.: US 2023/0346841 A1
(43) Pub. Date: Nov. 2, 2023

(54) COMPOSITIONS, METHODS AND USES FOR IN VITRO AND IN VIVO GENERATION OF FUNCTIONAL THYMIC TISSUE

(71) Applicant: THE REGENTS OF THE UNIVERSITY OF COLORADO, A BODY CORPORATE, Denver, CO (US)

(72) Inventors: Holger A. RUSS, Gainesville, FL (US); Stephan A. RAMOS, Aurora, CO (US); Antonio JIMENO, Greenwood Village, CO (US); John Jason MORTON, Thornton, CO (US)

(21) Appl. No.: 18/132,164

(22) Filed: Apr. 7, 2023

Related U.S. Application Data
(63) Continuation of application No. PCT/US2021/054059, filed on Oct. 7, 2021.
(60) Provisional application No. 63/088,904, filed on Oct. 7, 2020.

(51) Int. Cl. A61K 35/26; C12N 5/078; A61P 5/00; A61P 37/02

(52) U.S. Cl. CPC A61K 35/26; C12N 5/065; A61P 5/00; A61P 37/02; C12N 2502/1171; C12N 2506/45; C12N 2502/13

(57) ABSTRACT

Embodiments disclosed herein concern various cells, co-cultures, methods, systems, therapies, and treatments involving in vitro and in vivo generation and use of functional mammalian thymic tissue, thymus organs, and thymic organoids. In certain embodiments, cells, tissues, and organoids disclosed herein can be used to treat a subject having a thymic condition. In other embodiments, thymic cells and organoids produced by compositions and methods disclosed herein can be used to treat various conditions, diseases, and disorders, including auto-immune disorders, transplant rejections, cancer, or aging.

### PRIORITY

This application is a Continuation Application of International Application No. PCT/US2021/054059, filed on Oct. 7, 2021, which claims priority pursuant to U.S. 35 U.S.C. § 119(e) to U.S. Provisional Application No. 63/088,904 filed Oct. 7, 2020. These applications are incorporated herein by reference in their entirety for all purposes.

### GOVERNMENT FUNDING

This invention was made with government support under Grant Nos. R01CA213102-01A1 and R01CA149456-06A1 awarded by the National Institutes of Health. The government has certain rights in this invention.

### FIELD

Embodiments of the instant disclosure generally relate to compositions, methods, and systems for generating and using in vitro and in vivo generated thymic cells and thymic tissue for use in therapeutic applications and for the study of mammalian immune systems.

### BACKGROUND

The adaptive immune system plays a role in various processes such as cancer, organ transplantation, and autoimmunity. Thus, adaptive immunity has been the focus of many studies. However, studies focused on human T cell development and function are limited because studying a subject is confined to the use of mouse or xenogeneic models. While these studies have elucidated some mechanisms and aspects of human thymus development and function, there are limitations. There is a need for improved systems for studying adaptive immunity and for therapeutic interventions in health conditions that affect the thymus.

### SUMMARY

Embodiments of the instant disclosure generally relate to compositions, methods, and systems for generating and using in vitro and in vivo generated thymic cells and thymic tissue for use in therapeutic applications and for the study of mammalian immune systems. Certain embodiments disclosed herein concern compositions and methods for rapidly generating thymic cells and thymic tissues of use to treat medical conditions, generating improved animal models and for studying adaptive immunity.

In some embodiments, compositions, and methods for creating a plurality of robust thymic cells and thymic organoids having a variety of therapeutic applications. In certain embodiments, compositions include a mixture of a plurality of thymic cells including thymic stromal cells and thymic epithelial cells and a plurality of hematopoietic cells (HPCs) to generate target thymus cells of interest. In other embodiments, compositions disclosed herein include a mixture of a plurality of mammalian thymic cells including thymic stromal cells and thymic epithelial cells having one or more selected thymic stromal cell and/or thymic epithelial cell markers and a plurality of hematopoietic cells (HPCs). In some embodiments, compositions disclosed herein can have a plurality of mammalian thymic cells including thymic stromal cells and thymic epithelial cells having one or more thymic stromal cell and/or thymic epithelial cell markers including, one or more of PDGFRα (stromal cells) CD205, EPCAM, and FOXN1 (epithelial cells), In some embodiments, cells express of the population are about 70 to 90% EPCAM expressing, about 30 to 50% CD205, about 30 to 50% HOXA3 expressing cells. In other embodiments, compositions include a mixture of a plurality of mammalian thymic cells including thymic stromal cells and thymic epithelial cells expressing one or more of CD205, EPCAM, FOXN1, KRT8, KRT5, DLL1, DLL4, PD-L1, AIRE, HLA-DQ (class II), CD104, PAX9, SIX1, PSMB11, CCL21, CXCL12, RANK, CD80, CD86, Beta 5T, Tissues restricted antigens (TRAs) including but not limited to INS, IA2, GAD1, Myelin Basic Protein and Thyroglobulin and a plurality of HPCs. In certain embodiments, compositions disclosed herein include, but are not limited to a mixture of a plurality of mammalian thymic cells including thymic stromal cells and thymic epithelial cells and HPCs having one or more specified HPC markers including CD34 and CD45. HPCs can be obtained by isolated CD34+ cells from patients or cord blood or by differentiated of human pluripotent stem cells and/or stem cells into HPCs. In some embodiments, compositions disclosed herein include a plurality of mammalian HPCs expressing one or more marker including, but not limited to, CD34 and CD45, CD5, CD7, CD43, CD3, TCRa/b, CD4, CD8, PD1, NCAM+, CD34+, VECAD+, CD90+, CD27, CD38, CD43, CD48, CD117, Sca-1 and CD150. In accordance with these embodiments, incubation of the above referenced mixtures of cells generates AIRE positive thymic cells for therapeutic use and further study.

In some embodiments, compositions herein can further include a plurality of mesenchymal cells. In some embodiments, compositions herein can further have a plurality of mesenchymal cells wherein the mesenchymal cells can express PDGFRα. In other embodiments, compositions herein can further include a plurality of mesenchymal cells expressing one or more of CD105, CD90, CD73, VIM, PDGFRb, FGF7, FGF10 and TE-7 or all six markers.

In other embodiments, compositions include a mixture of a plurality of mammalian thymic cells including thymic stromal cells and thymic epithelial cells and a plurality of HPCs.

In some embodiments, compositions herein can have a plurality of mammalian thymic cells encompassing thymic stromal cells and thymic epithelial cells and a plurality of mesenchymal cells. In some embodiments, compositions herein can have a plurality of mammalian thymic cells encompassing thymic stromal cells and thymic epithelial cells EPCAM, CD205 and FOXN1a plurality of mesenchymal cells PDGRFα. Mesenchymal cells can be obtained by isolation from human tissue samples, including human thymi, or by differentiation of human pluripotent stem cells and/or stem cells into mesenchymal cells, including splanchnic mesenchyme.

In some embodiments, compositions herein include a mixture of a plurality of mammalian thymic cells including thymic stromal cells and thymic epithelial cells and a plurality of mesenchymal cells and further include HPCs contemplated herein. In certain embodiments, compositions disclosed herein can have a plurality of mammalian thymic cells including thymic stromal cells and thymic epithelial cells having markers disclosed herein, a plurality of mesenchymal cells having markers disclosed herein and further include HPCs for rapid generation of targeted thymus cells of use for therapeutic applications.

In some embodiments, compositions including a plurality of HPCs can further include HPCs having one or more HPCs markers such as CD34, CD45 positive HPC cells. In some embodiments, compositions herein can further have a plurality of HPCs expressing one or more marker including, but not limited to, CD34 and CD45, CD5, CD7, CD43, CD3, TCRa/b, CD4, CD8, and PD1, NCAM+, CD34+, VECAD+, and CD90+, CD27, CD38, CD43, CD48, CD117, Sca-1 and CD150.

In some embodiments, compositions including mixtures of cells indicated in the paragraphs above can further contain thymus cells expressing one or more of CD205, EPCAM, FOXN1, KRT8, KRT8, DLL1, DLL4, PD-L1, AIRE, HLA-DQ (class II), CD104, PAX9, SIX1, PSMB11, CCL21, CXCL12, RANK, CD80, CD86, Beta 5T, Tissues restricted antigens (TRAs) including but not limited to INS, IA2, GAD1, Myelin Basic Protein and Thyroglobulin, PDGFRα, CD34 and CD45, CD5, CD7, CD43, CD3, TCRa/b, CD4, CD8, PD1, CD125 NCAM+, CD34+, VECAD+, and CD90+, CD105, CD90, CD73, VIM, PDGFRb, CD27, CD38, CD43, CD48, CD117, Sca-1 and CD150, CD105, CD90, CD73, VIM, PDGFRb, FGF7, FGF10, TE-7 over the course of differentiation. In some embodiments, compositions herein can express one or more CD205, EPCAM, FOXN1, KRT8, KRT8, DLL1, DLL4, PD-L1, AIRE, HLA-DQ (class II), Tissues restricted antigens (TRAs) including but not limited to INS, IA2, GAD1, Myelin Basic Protein and Thyroglobulin, PDGFRα, CD34 and CD45, CD5, CD7, CD43, CD3, TCRa/b, CD4, CD8, PD1, CD125 NCAM+, CD34+, VECAD+, and CD90+, CD105, CD90, CD73, VIM, PDGFRb, CD27, CD38, CD43, CD48, CD117, Sca-1 and CD150, CD105, CD90, CD73, VIM, PDGFRb, FGF7, FGF10, TE-7 after about 0 days to about 30 days during course of differentiation of mixtures of cells populations and incubation thereof.

In certain embodiments, compositions containing mixtures of cells disclosed herein as indicated at least in paragraph [0009] above can be incubated for a period of time in order to generate thymus cells expressing at least the cell marker AIRE protein after about 1 hour to about 1 day or less than 30 days of accelerated and improved differentiation. In other embodiments, compositions containing mixtures of cells disclosed herein as indicated at least in paragraph [0009] above can be incubated for a period of time in order to generate CD4+/CD8+ T cells after about 1 hour of incubation to about 30 days of accelerated and improved differentiation. In certain embodiments, a combination of mammalian thymic cells including thymic stromal cells and thymic epithelial cells, a plurality of mesenchymal cells and HPCs provide for a more rapid differentiation and production of desirable cells containing these markers.

In some embodiments, thymus cells and thymic tissues generated by compositions and methods disclosed herein can be harvested from a differentiated culture (after a pre-determined differentiation period) to be used immediately, after a day or several days in a therapeutic application. In some embodiments, compositions herein can be harvested from the culture and optionally purified immediately before use in a therapeutic application. In other embodiments, thymic cells and/or thymic tissues generated by compositions and methods disclosed herein can be harvested and stored at about −80° C. to about 4° C. for a period of time before use in a therapeutic setting. In some embodiments, thymic cells and/or thymic tissues generated by compositions and methods disclosed herein can be harvested and stored at about −80° C. to about 4° C. (refrigeration or on ice or dry ice) for about a day to about a year before use in a therapeutic application. In some embodiments, thymic cells and/or thymic tissues generated by compositions and methods disclosed herein can be harvested and subjected to at least one freeze-thaw cycle before use in a therapeutic application. In certain embodiments, thymic cells and/or thymic tissues generated by compositions and methods disclosed herein can be harvested by methods known in the art and later delivered for therapeutic use or stored at reduced temperatures for later use or cell expansion, such as culturing.

In some embodiments, the present disclosure provides compositions and methods for generating differentiated thymic epithelial cells expressing specific markers. In some embodiments, compositions and methods disclosed herein provide for improved production and differentiation into desirable T cell populations such as production of thymic epithelial cells expressing AIRE. In some embodiments, methods disclosed herein concern mixing populations in culture of a plurality of mammalian thymic cells including thymic stromal cells and thymic epithelial cells, a plurality of mesenchymal cells and a plurality of HPCs and incubating the mixture for a period of time and generating thymic epithelial cells expressing AIRE as a marker where the mixed population is incubated for about 1 week to about 3 months. In some embodiments, compositions, and methods for generating thymic epithelial cells expressing AIRE can include incubating a population of thymic stromal cells and thymic epithelial cells disclosed herein with a plurality of mesenchymal cells disclosed herein. In other embodiments, compositions and methods for generating thymic epithelial cells expressing AIRE can include incubating a population of a plurality of HPCs disclosed herein with a plurality of mesenchymal cells disclosed herein. In yet other embodiments, compositions and methods for generating thymic epithelial cells expressing AIRE can include incubating a population of thymic stromal cells and thymic epithelial cells, a plurality of HPCs disclosed herein and a plurality of mesenchymal cells disclosed herein for a period of time.

In some embodiments, the present disclosure provides methods for treating a subject having or suspected of having a health condition concerning the thymus or adaptive immunity. In other embodiments, compositions and methods for treating a subject having or suspected of having a health condition concerning the thymus or adaptive immunity can include delivering a population of thymic epithelial cells or thymic organoids created by methods disclosed herein to the subject. In accordance with these embodiments, cells or organoids can be harvested, prepared and transported for administration to the subject by any means known in the art (e.g. bolus administration or infusion or transplantation).

In other embodiments, the present disclosure provides kits for storing, transporting or practicing one or more of the compositions and methods disclosed herein. In some embodiments, kits can include one or more of cell populations or mixture of cell populations or products produced by methods disclosed herein. In other embodiments, kits disclosed herein can one or more of cell populations or mixture of cell populations or products produced by methods disclosed herein and at least one container.

In some embodiments, the present disclosure provides animal models for assessing therapies to treat a health condition or for producing an expanded population of cells such as humanized cell populations. In some embodiments, animal models can include an in vivo screening platform for one or more therapies to treat a health condition. In some embodiments, animal models herein can be in vivo screening platforms for one or more chemical-based and/or immuno-based therapies to treat a health condition. In some embodiments, animal models herein can be used for assessing therapies to treat a cancer, a thymic condition, and immune deficiency, a viral infection, and/or a bacterial infection and/or aging such as aging immunity.

In certain embodiments, health conditions contemplated herein to be treated by cell populations and/or organoids produced herein can include, but are not limited to, a disorder of the thymus. In accordance with these embodiments, a disorder of a thymus can include, but it not limited to, an ablated, missing, malfunctioning, injured or other thymus condition. In other embodiments, thymic cells and/or thymic organoids generated herein can be patient specific or patient typed (e.g. donor screened) using patient derived or donor screened cell cultures.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

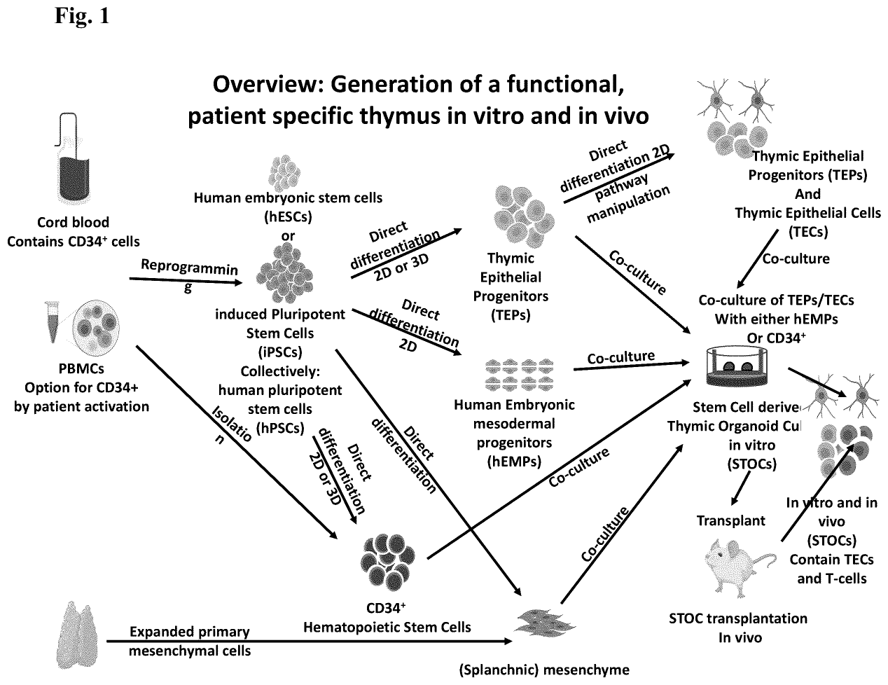

FIG. 1 represents an exemplary experiment of the instant disclosure illustrating a schematic diagram of depicting methods for the generation of functional thymus organ using the stem cell derived thymic organoid cultures (STOC) in accordance with certain embodiments of the present disclosure.

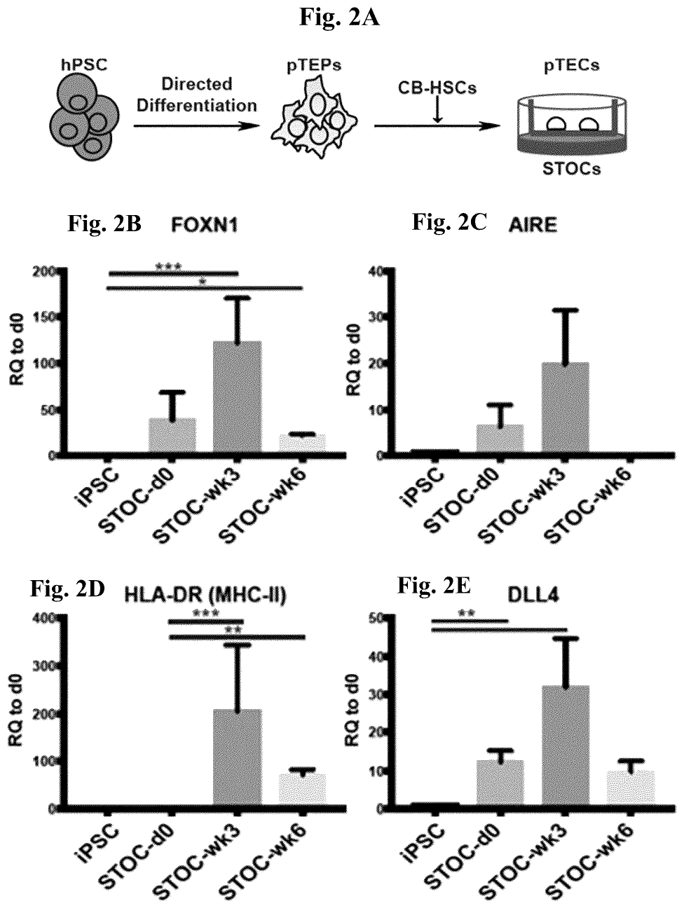

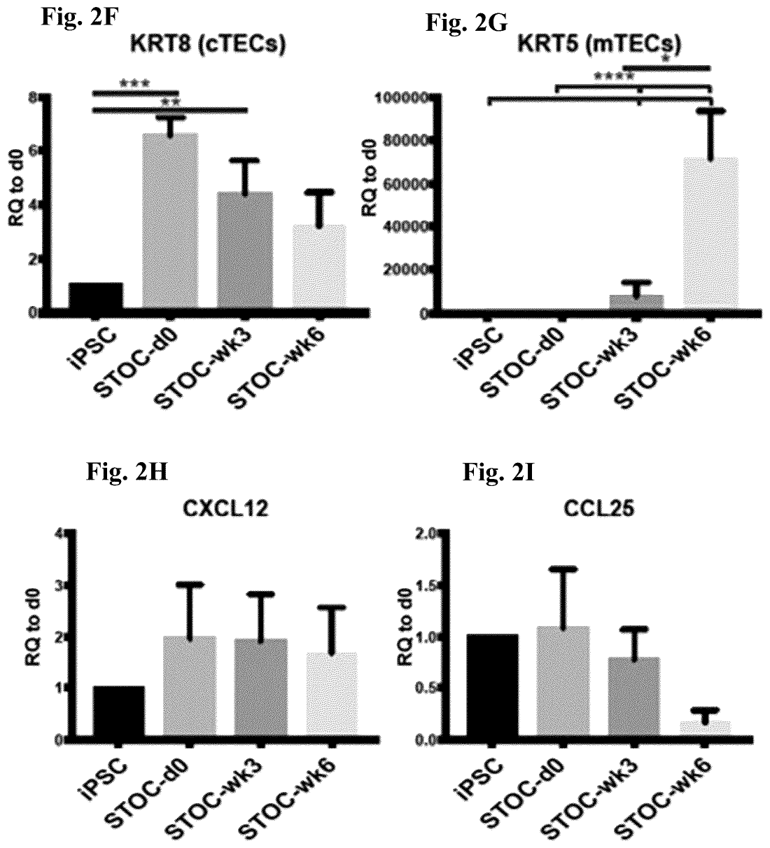

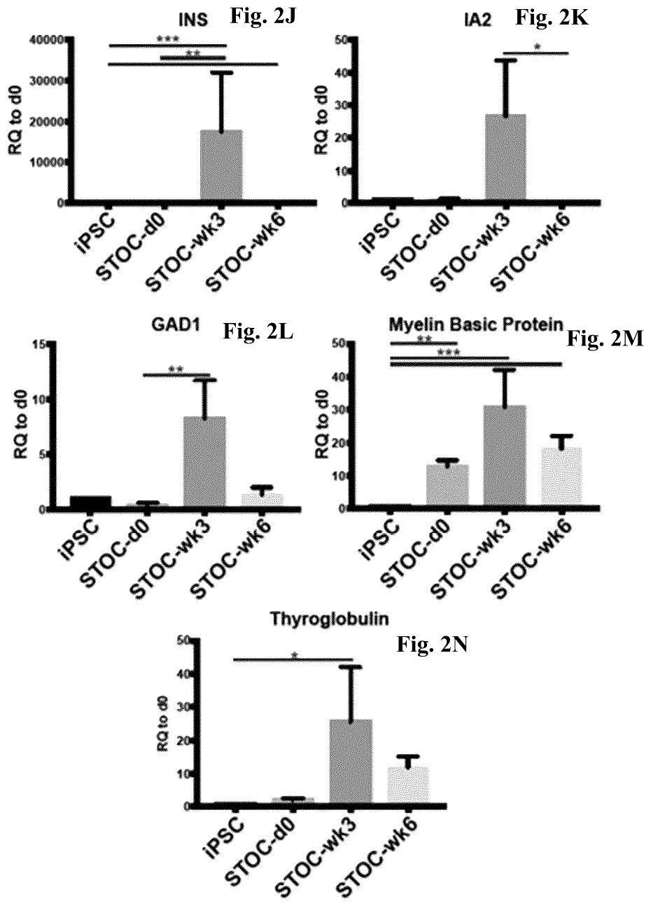

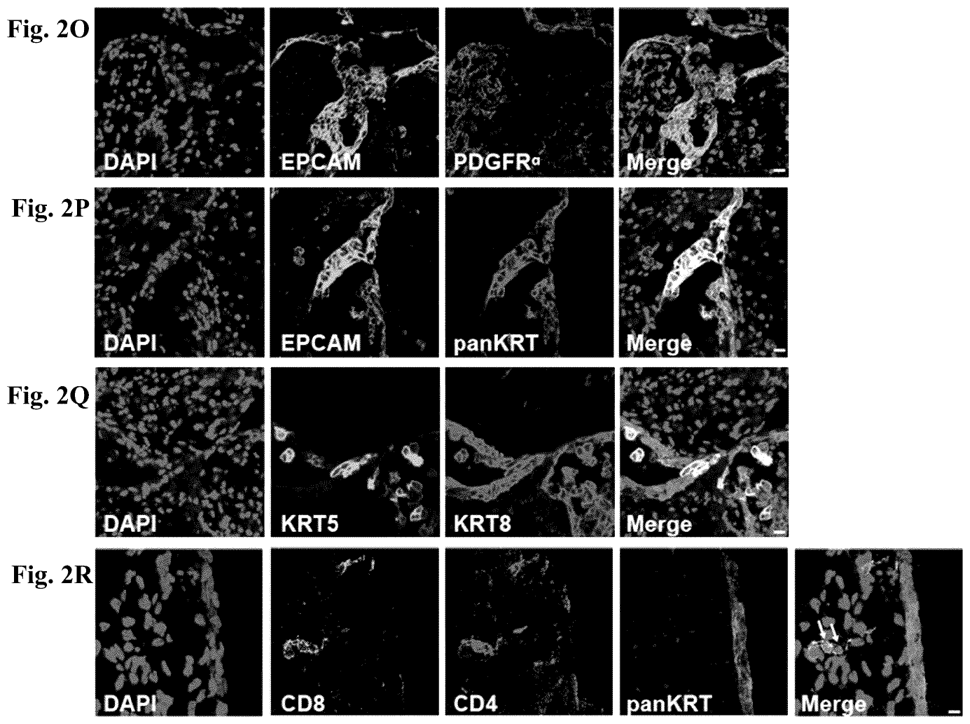

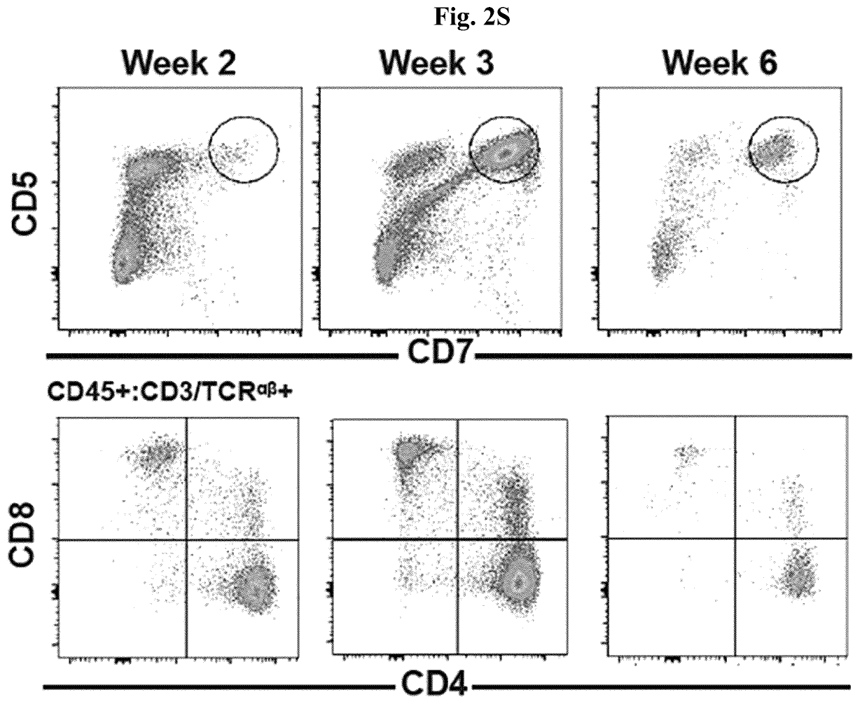

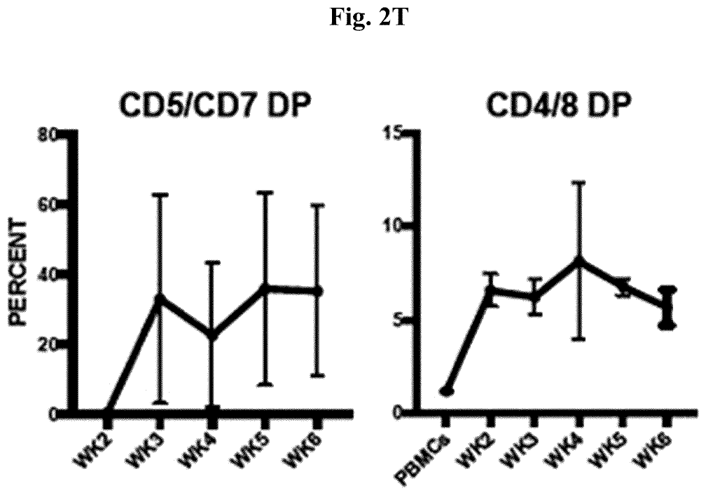

FIGS. 2A-2T represent exemplary experiments of the instant disclosure illustrating in vitro generation and characterization of patient specific, human iPSC-derived TECs in accordance with certain embodiments of the present disclosure. FIG. 2A illustrates a schematic of the exemplary experiment. FIGS. 2B-2N illustrate graphs depicting gene expression levels in STOCs over time. FIGS. 20-2R illustrate immunofluorescence (IF) straining of STOCs for thymic cell markers. FIG. 2S illustrates representative scatter plots of STOCs over time. FIG. 2T illustrates a graph depicting quantification of flow cytometry experiments.

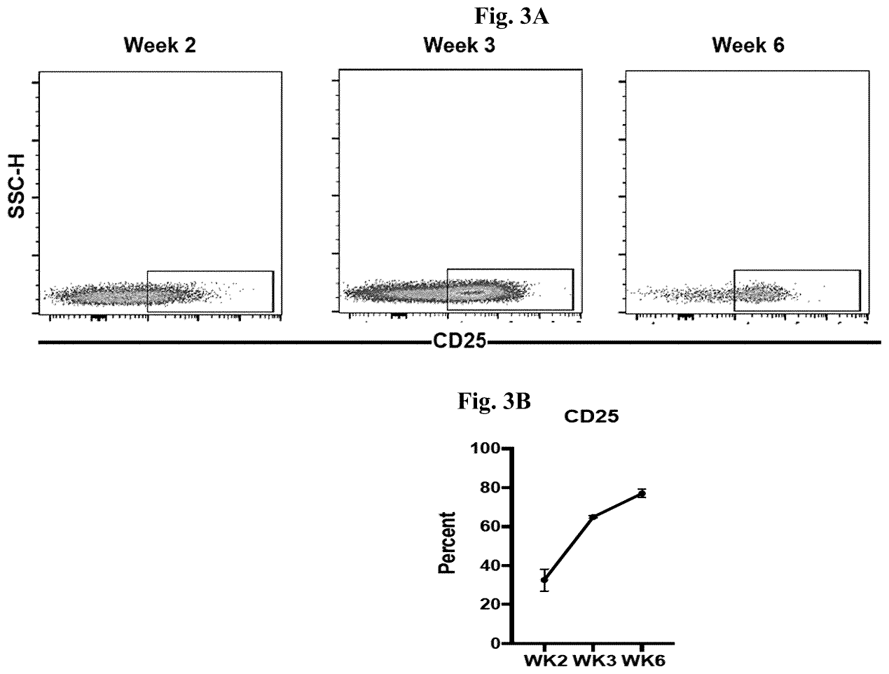

FIGS. 3A-3B represent exemplary experiments of the instant disclosure illustrating activation of carrier T cells in allogenic STOCs in accordance with certain embodiments of the present disclosure. FIG. 3A illustrates representative scatter plots of STOCs over time and FIG. 3B illustrates a graph depicting quantification of flow cytometry experiments.

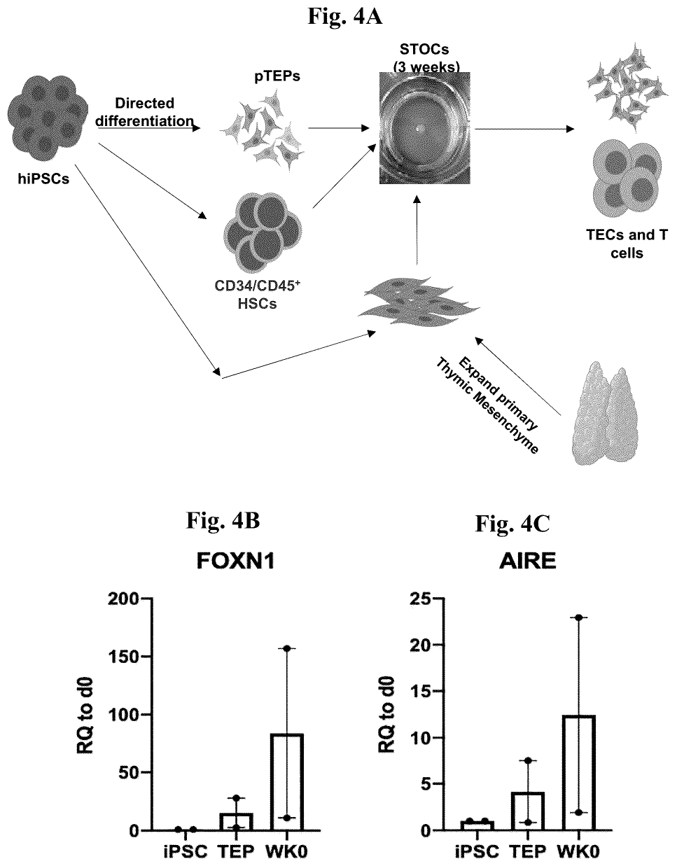

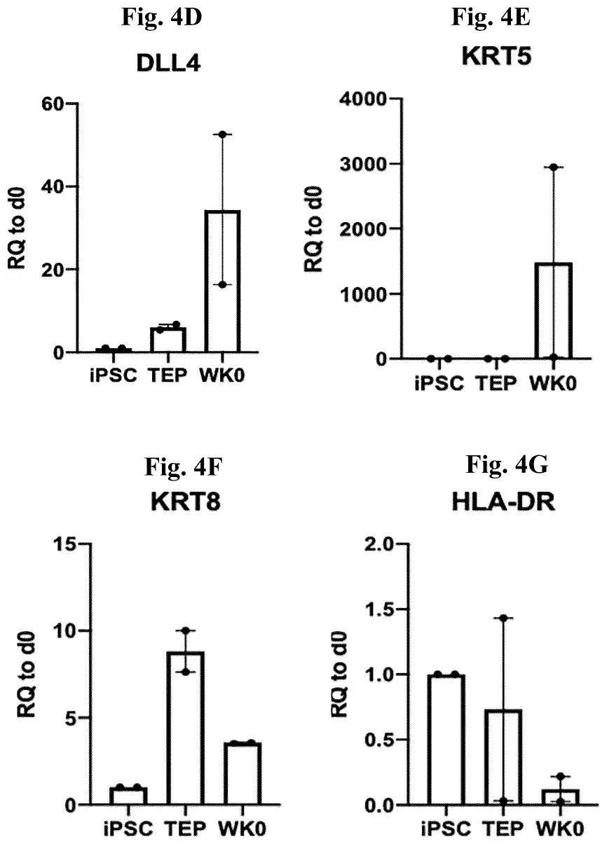

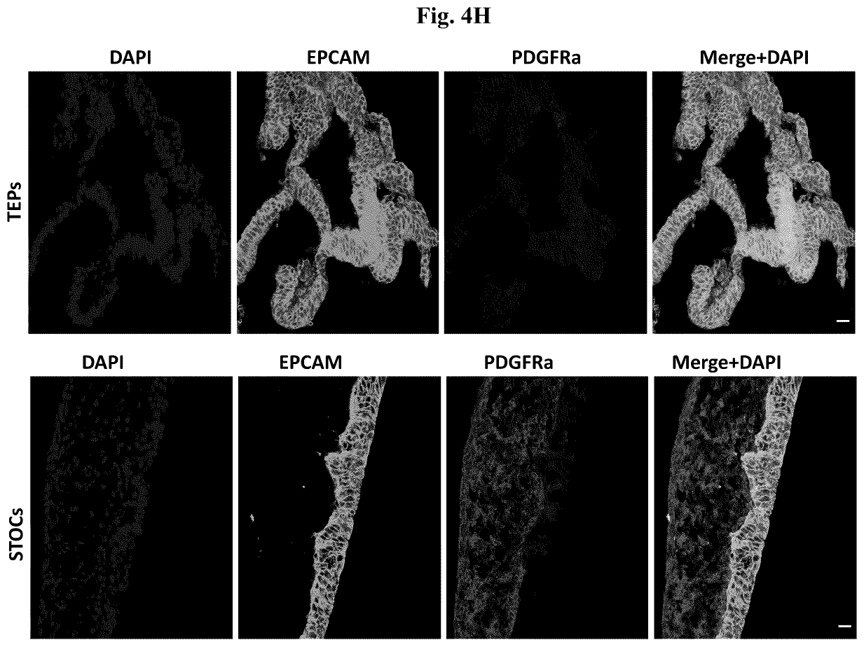

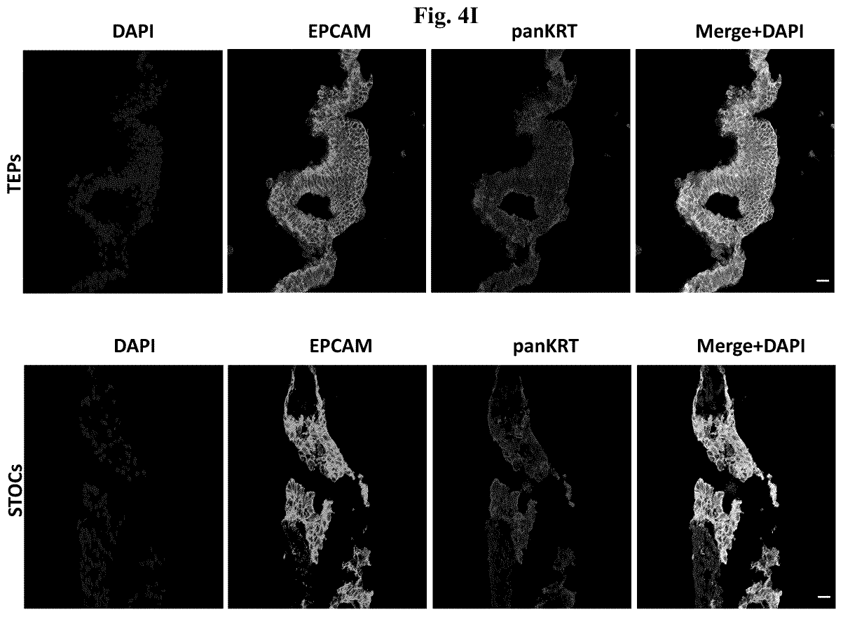

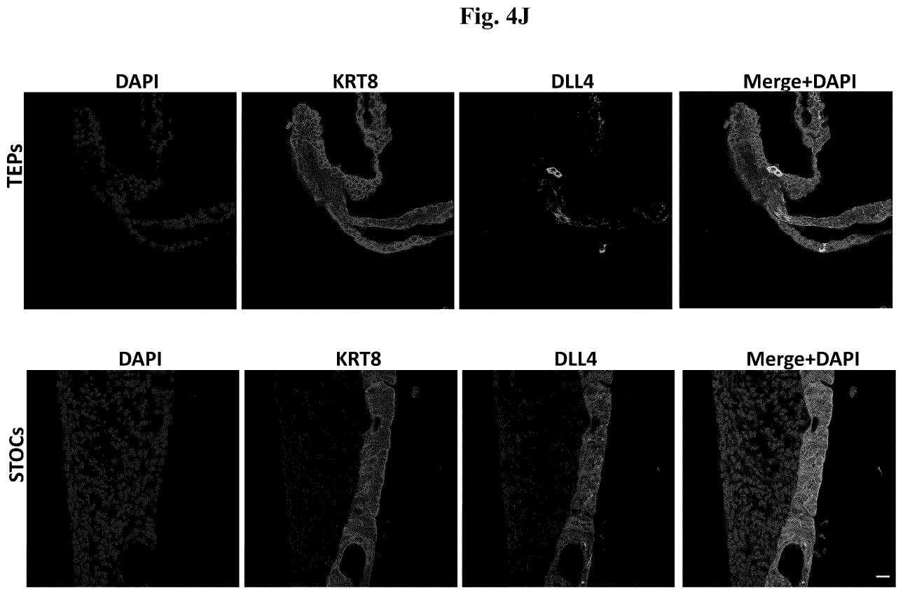

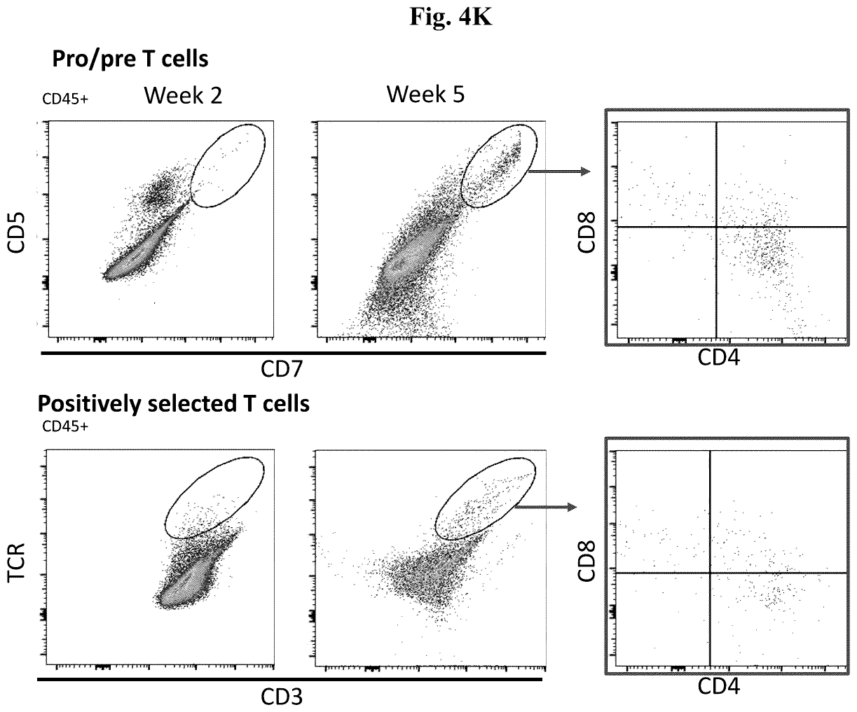

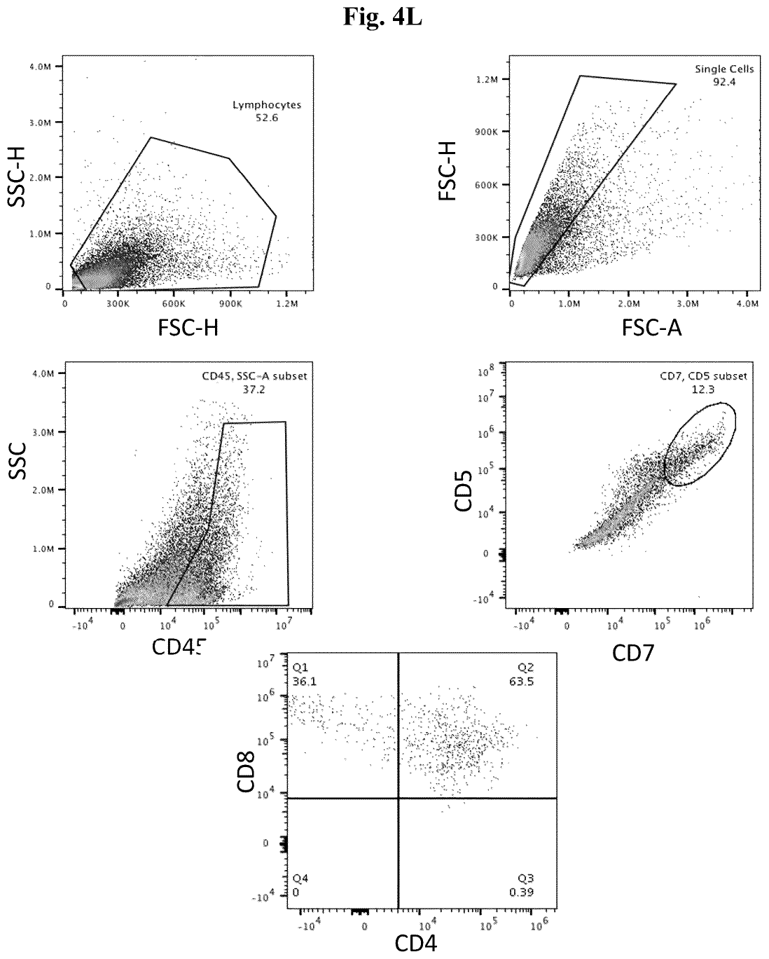

FIGS. 4A-4L represent exemplary experiments of the instant disclosure illustrating generation and characterization of isogenic STOCS that illustrate functional thymus differentiation and facilitate T cell development in accordance with certain embodiments of the present disclosure. FIG. 4A illustrates a schematic of the exemplary experiment. FIGS. 4B-4G illustrate graphs depicting gene expression levels in STOCs over time. FIGS. 4H-4J illustrate IF straining of STOCs for thymic cell markers. FIGS. 4K-4L illustrate representative scatter plots of STOCs

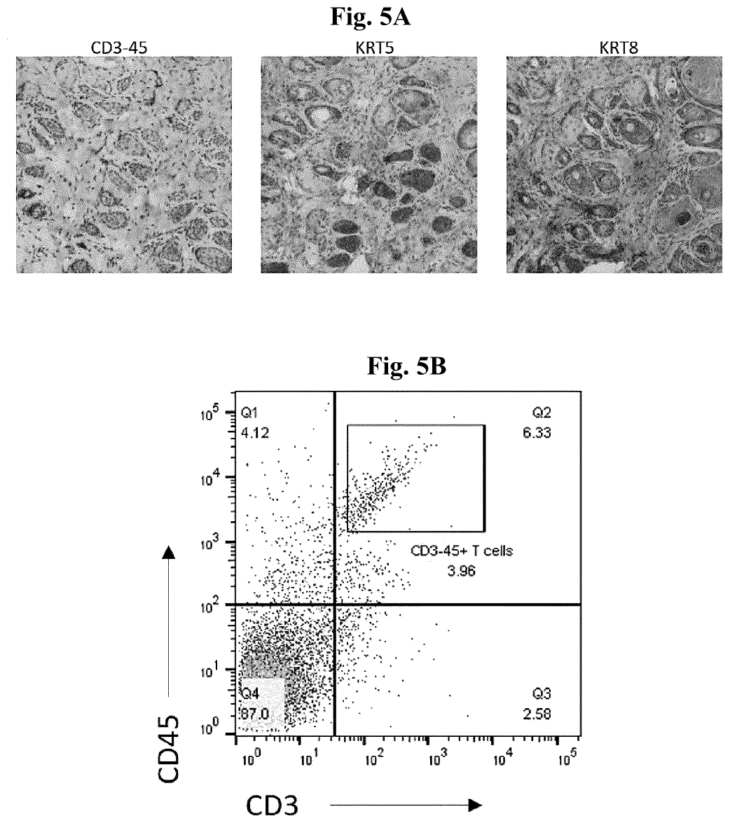

FIGS. 5A-5B represent exemplary experiments of the instant disclosure illustrating transplanted isogenic STOCs having thymic structures and human T cells in the periphery in accordance with certain embodiments of the present disclosure. FIG. 5A illustrates representative images of immunohistochemistry (IHC) staining of transplanted isogenic STOCs. FIG. 5B illustrates a representative scatter plot of isolated cells.

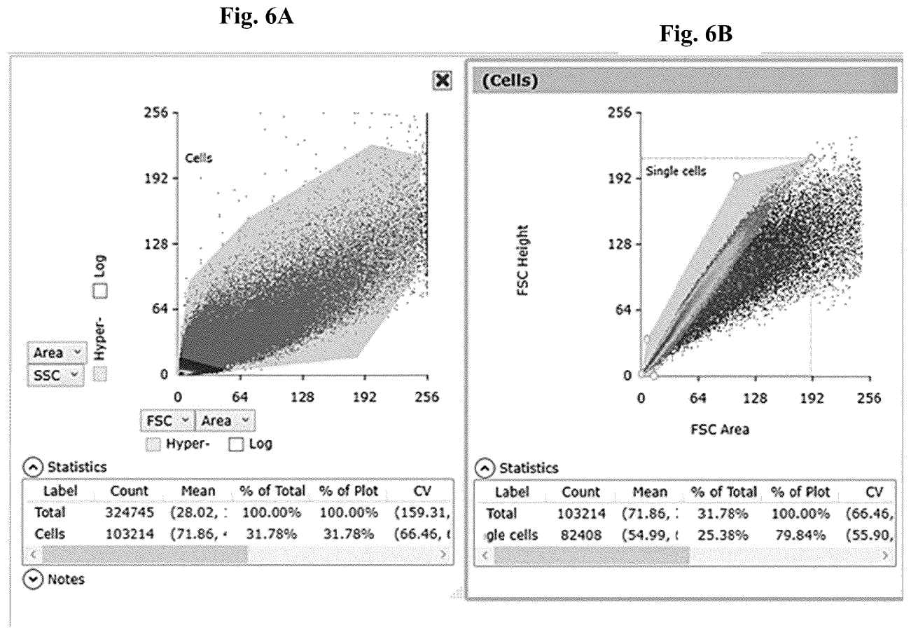

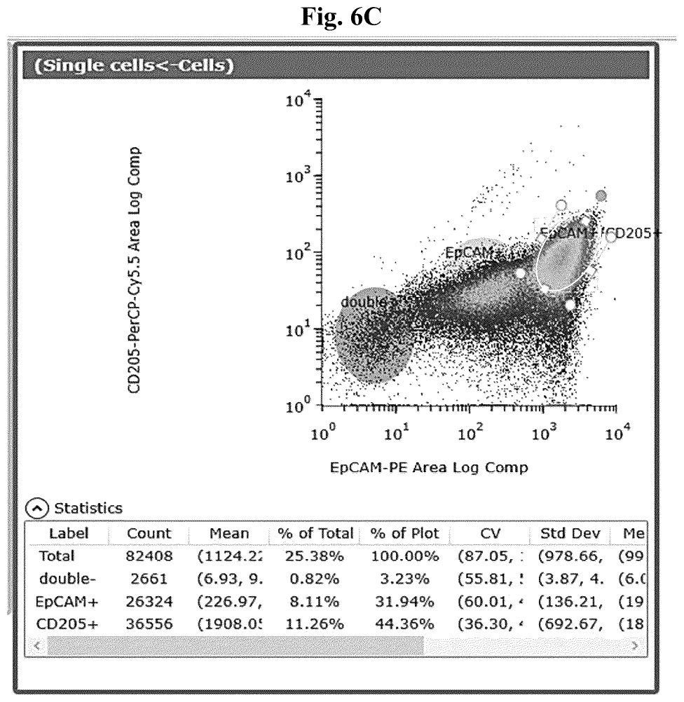

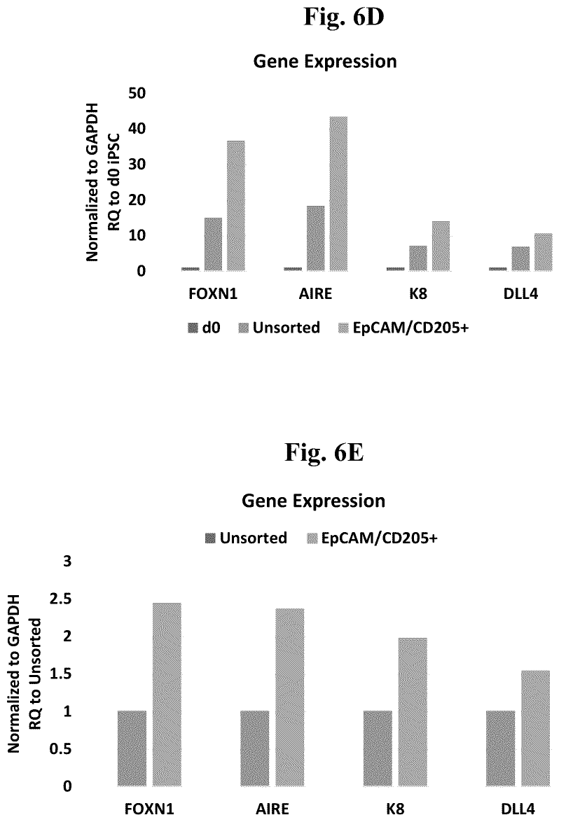

FIGS. 6A-6E represent exemplary experiments of the instant disclosure illustrating isolation and characterization of CD205-enriched human thymic cells in accordance with certain embodiments of the present disclosure. FIGS. 6A-6C illustrate representative scatter plots of human thymic cells. FIGS. 6D-6E illustrate graphs depicting gene expression levels in human thymic cells.

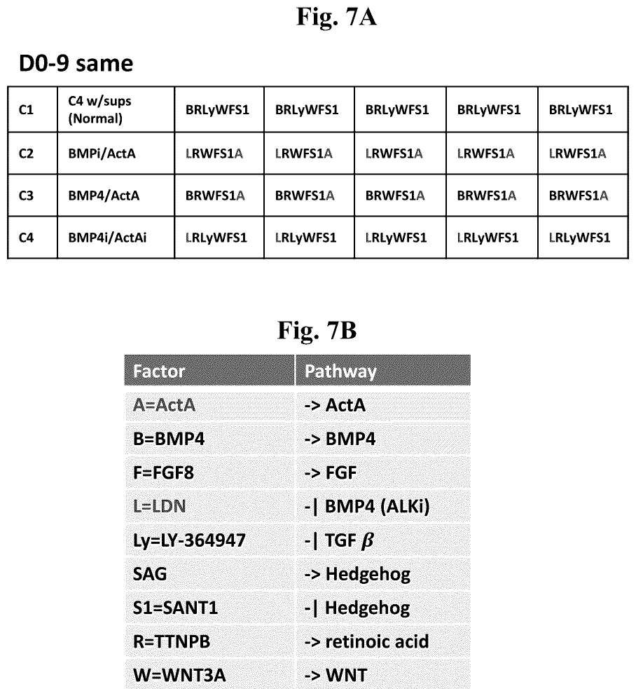

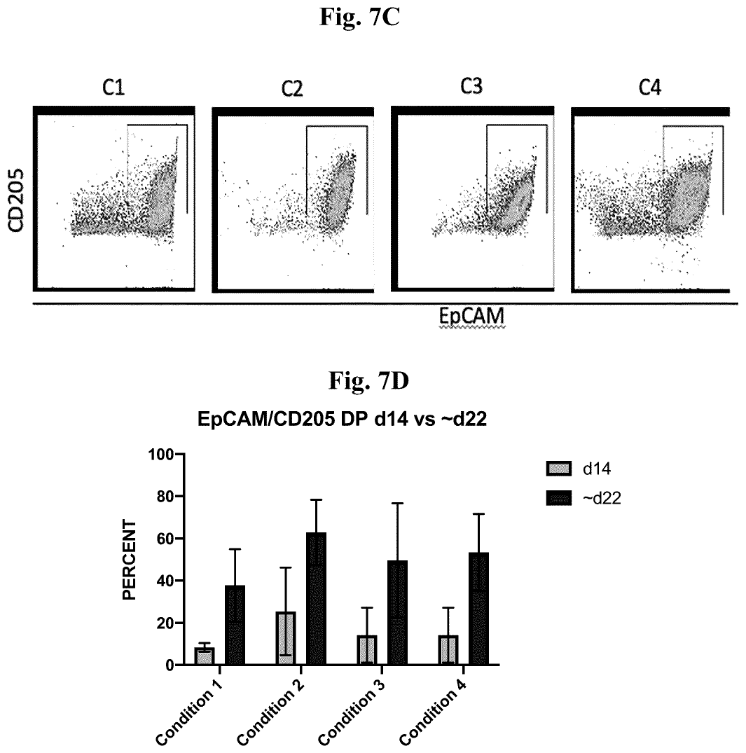

FIGS. 7A-7D represent exemplary experiments of the instant disclosure illustrating Activin A induction of thymic cell generation in accordance with certain embodiments of the present disclosure. FIGS. 7A-7B illustrate images depicting details of the exemplary method. FIG. 7C illustrates representative scatter plots of differentiated hPSC cells. FIG. 7D illustrates a graph depicting quantification of the percentage of EpCAM/CD205 positive cells.

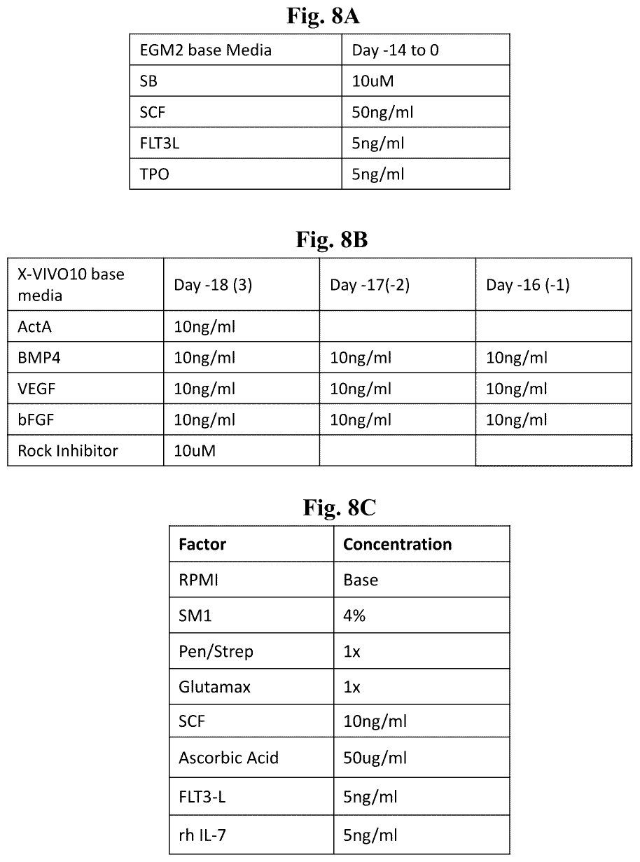

FIGS. 8A-8C represent exemplary experiments of the instant disclosure illustrating a materials and methods of generating hEMP cells in accordance with certain embodiments of the present disclosure.

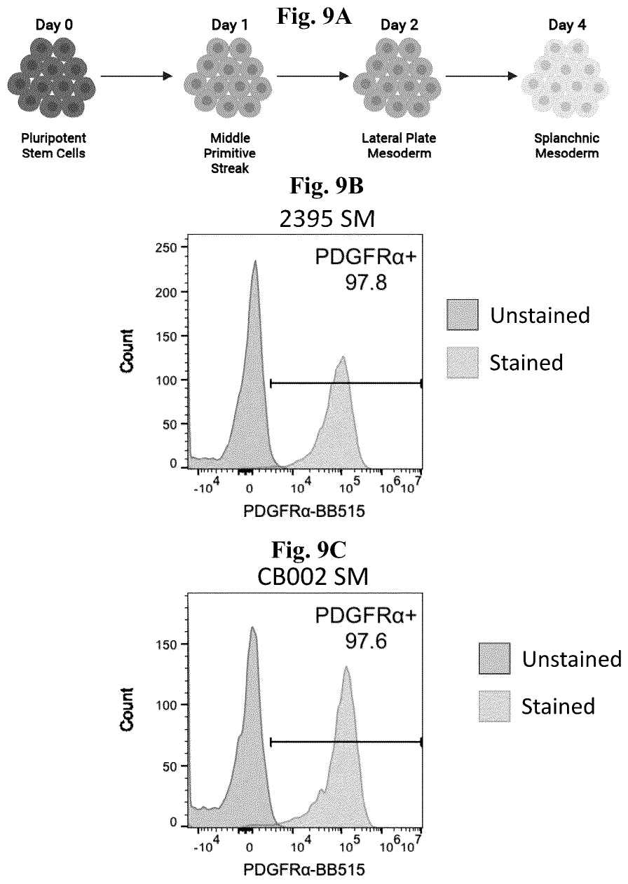

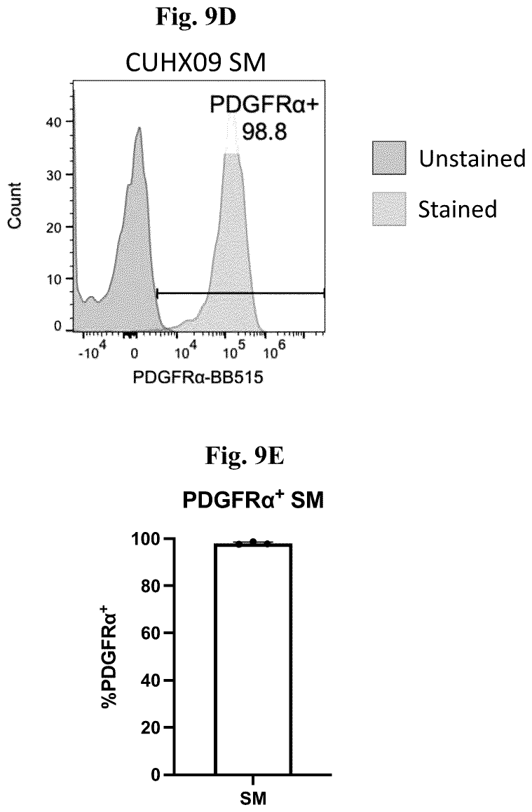

FIGS. 9A-9E represent exemplary experiments of the instant disclosure illustrating generation and characterization of splanchnic mesenchyme in accordance with certain embodiments of the present disclosure. FIG. 9A illustrates a schematic of the exemplary experiment. FIGS. 9B-9D illustrate graphs depicting representative FACS of splanchnic mesenchyme. FIG. 9E illustrates a graph depicting quantification of the percentage of PDGFRα positive cells.

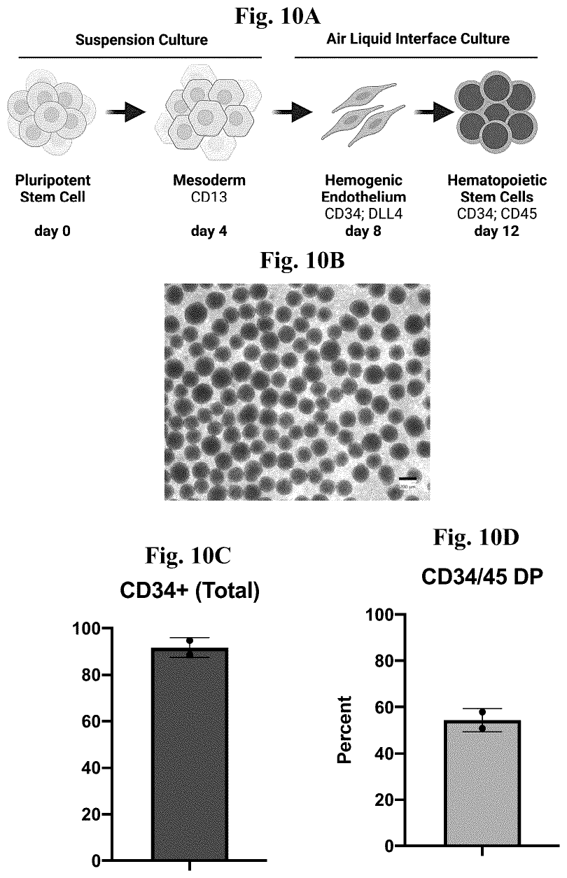

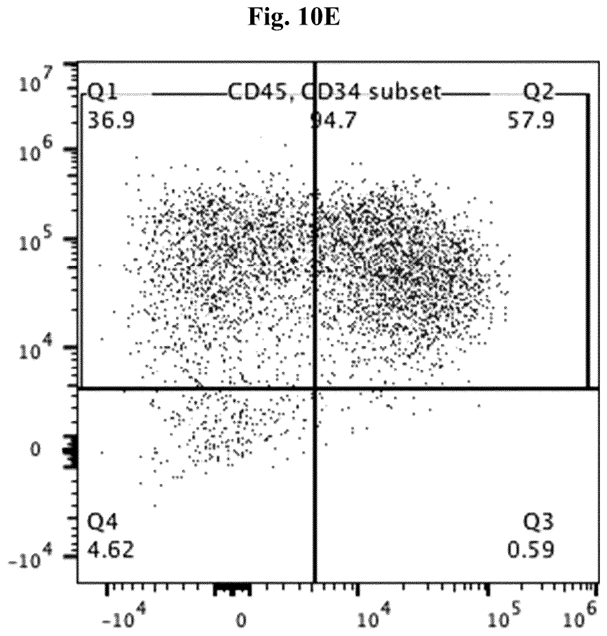

FIGS. 10A-10E represent exemplary experiments of the instant disclosure illustrating generation and characterization of hematopoietic stem cells/T-cell progenitor cells in accordance with certain embodiments of the present disclosure. FIG. 10A illustrates a schematic of the exemplary experiment. FIG. 10B illustrates a representative image of human pluripotent stem cell clusters at the start of the differentiation protocol. FIGS. 10C-10D illustrate graphs depicting quantification of FACS analysis on differentiated cells. FIG. 10E illustrates a representative scatter plot of differentiated cells.

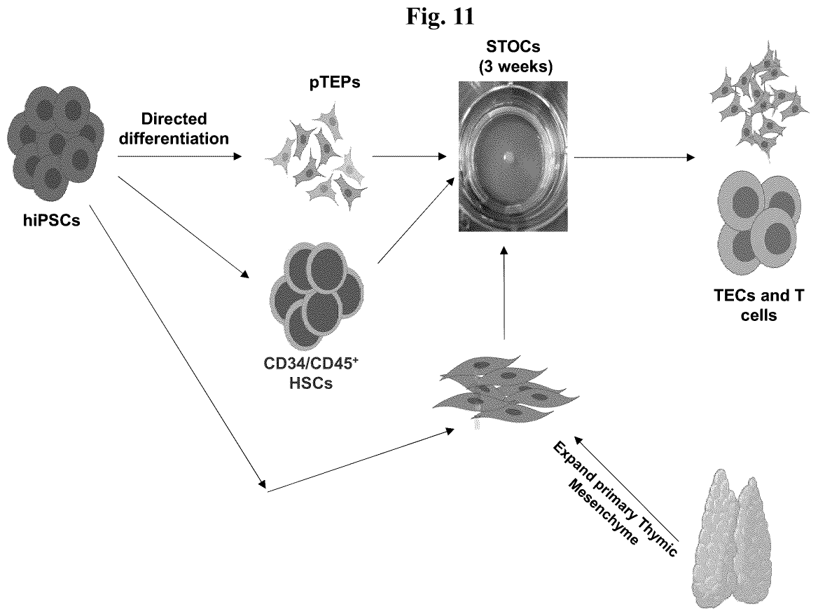

FIG. 11 represents an exemplary experiment of the instant disclosure illustrating a schematic diagram of depicting methods for the generation stem cell derived thymic organoid cultures (STOC) in accordance with certain embodiments of the present disclosure.

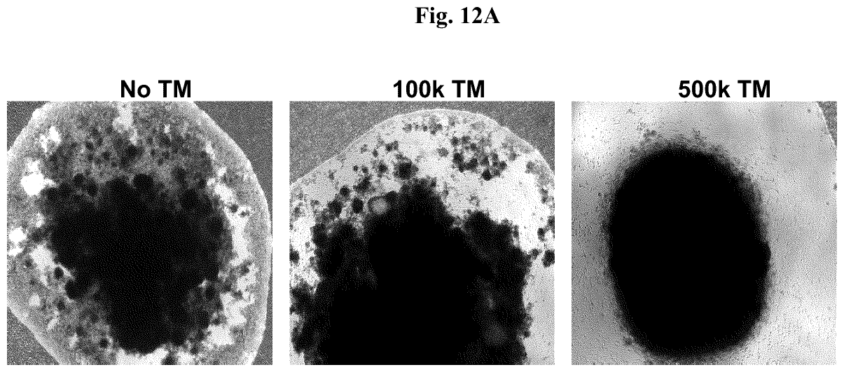

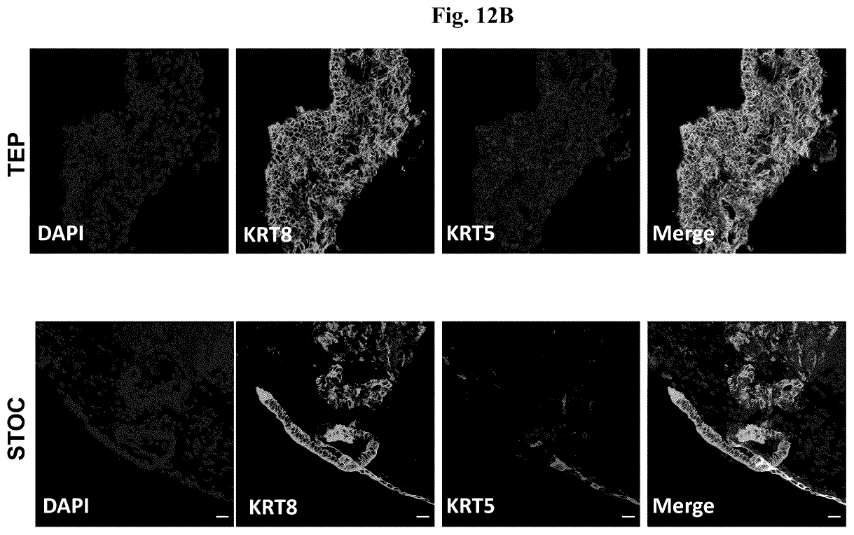

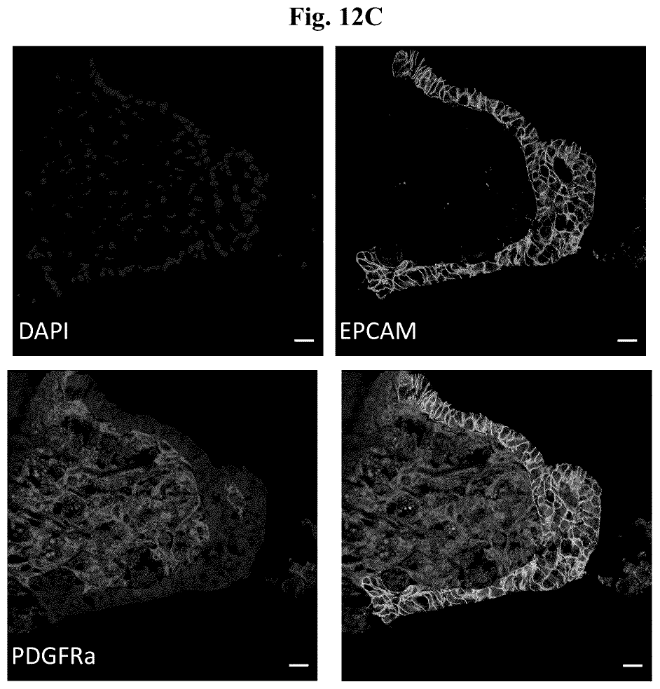

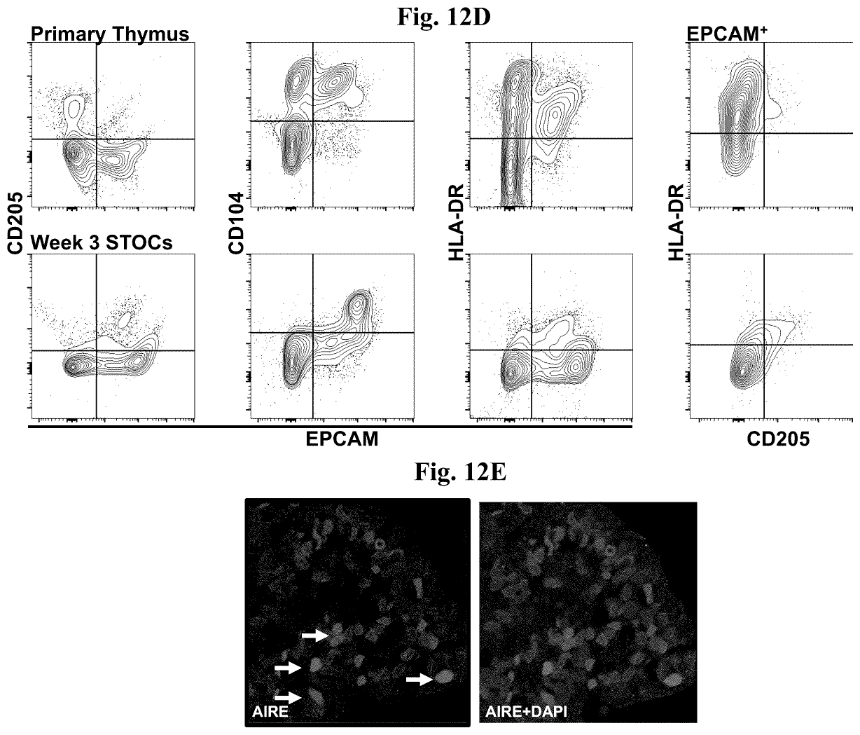

FIGS. 12A-12E represent exemplary experiments of the instant disclosure illustrating generation of TECs using STOCs consisting of TEPs, HSCs/T cell progenitors, and mesenchymal cells and characterization thereof in accordance with certain embodiments of the present disclosure. FIG. 12A illustrates representative images of STOCs with and without mesenchyme.

FIGS. 12B-12C illustrate IF straining of STOCs for thymic cell markers. FIG. 12D illustrates representative scatter plots of STOCs. FIG. 12E illustrates representative images of IF straining of STOCs for AIRE expression.

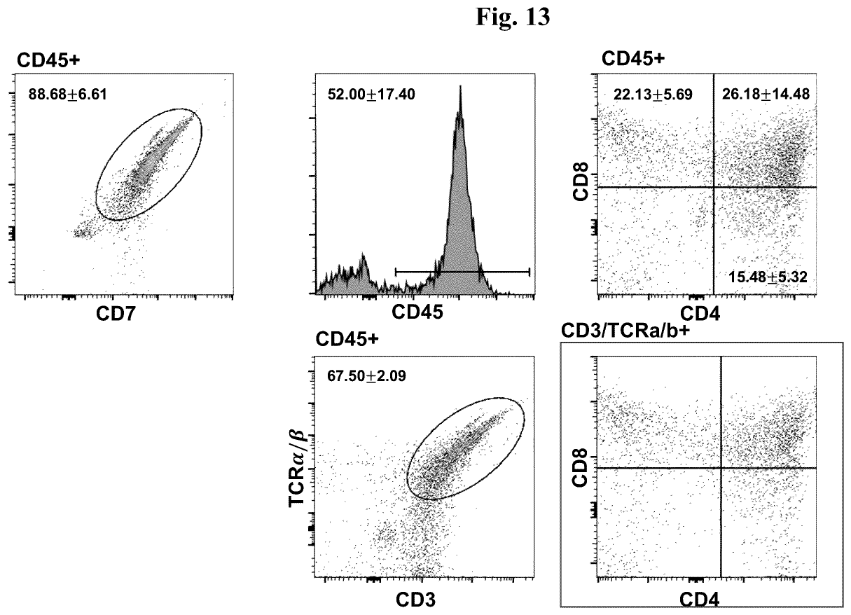

FIG. 13 represents an exemplary experiment of the instant disclosure illustrating improved generation of single positive CD4 and CD8 positive T cells in STOCs in accordance with certain embodiments of the present disclosure.

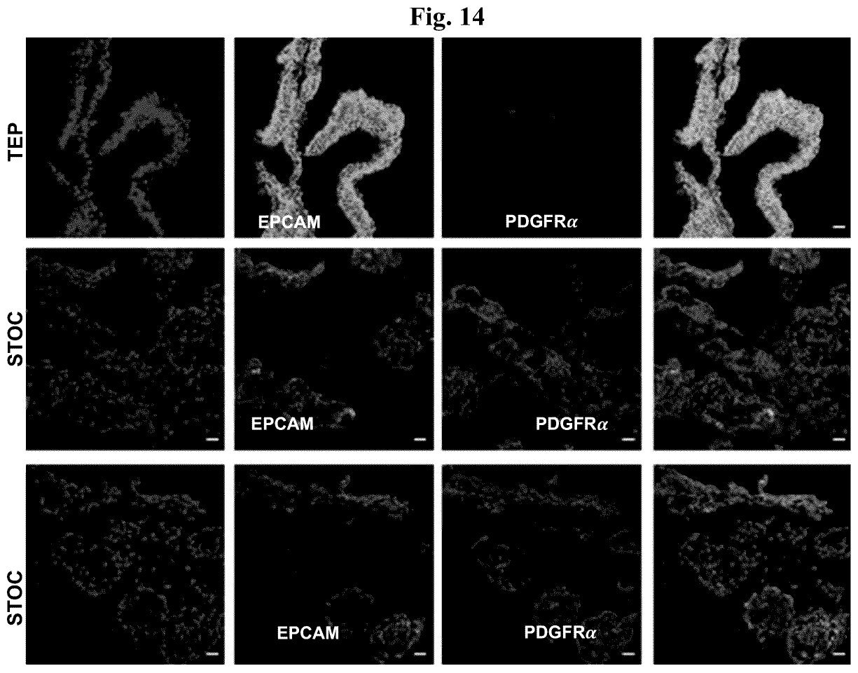

FIG. 14 represents an exemplary experiment of the instant disclosure illustrating spontaneous generation of mesenchyme in STOCs initially created without mesenchyme in accordance with certain embodiments of the present disclosure.

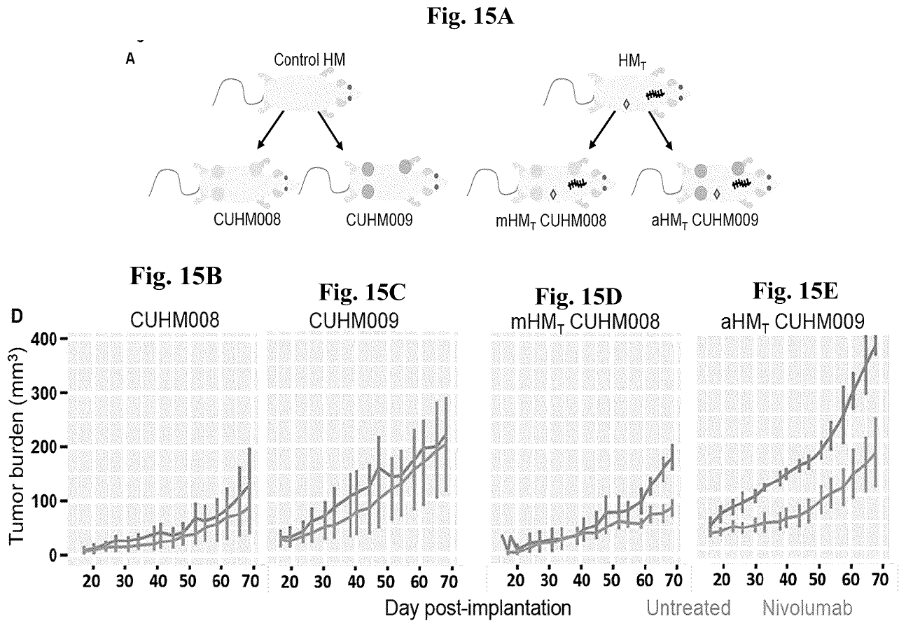

FIGS. 15A-15E represent exemplary experiments of the instant disclosure illustrating tumor burden of unmodified humanized mice or thymectomized humanized mice in accordance with certain embodiments of the present disclosure. FIG. 15A illustrates a schematic of the exemplary experiment. FIGS. 15B-15E illustrate graphs of tumor burden in mice treated with placebo or nivolumab over time after tumor cell implantation

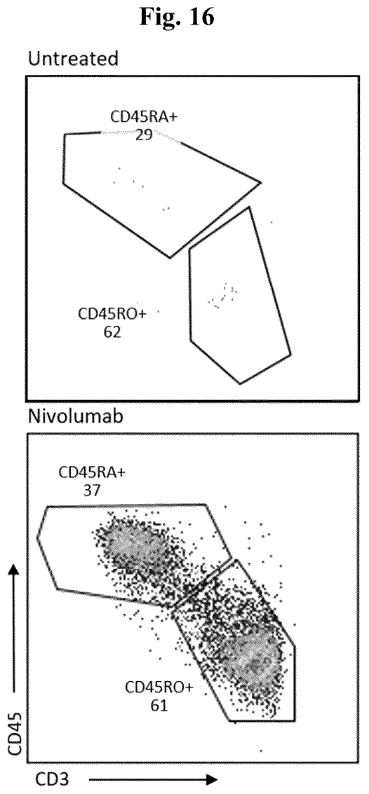

FIG. 16 represents an exemplary experiment of the instant disclosure illustrating cytometric identification of CD45RA+ (naïve) and CD45RO+ (memory) T cells isolated from untreated and nivolumab-treated aHM_{T} spleens in accordance with certain embodiments of the present disclosure.

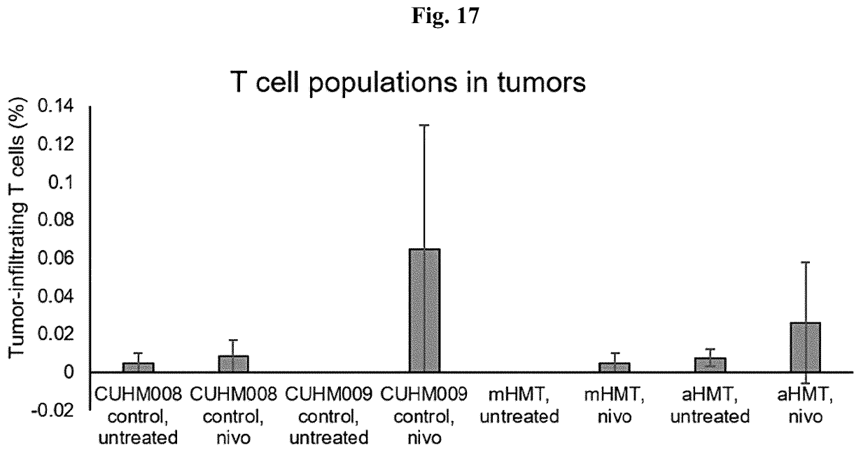

FIG. 17 represents an exemplary experiment of the instant disclosure illustrating quantification of infiltrative T cell populations in tumors collected from all unmodified humanized mice or thymectomized humanized mice after treatment with placebo or nivolumab in accordance with certain embodiments of the present disclosure. in accordance with certain embodiments of the present disclosure.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

In some embodiments, compositions, and methods for creating a plurality of robust thymic cells and thymic organoids having a variety of therapeutic applications. In certain embodiments, compositions include a mixture of a plurality of thymic cells including thymic stromal cells and thymic epithelial cells and a plurality of hematopoietic cells (HPCs) to generate target thymus cells of interest. In other embodiments, compositions disclosed herein include a mixture of a plurality of mammalian thymic cells including thymic stromal cells and thymic epithelial cells having one or more selected thymic stromal cell and/or thymic epithelial cell markers and a plurality of hematopoietic cells (HPCs) In certain embodiments, these cells express PDGFRα (stromal cells) and/or CD205, EPCAM, and FOXN1 (epithelial cells). In other embodiments, compositions include a mixture of a plurality of mammalian thymic cells including thymic stromal cells and thymic epithelial cells expressing one or more of CD205, EPCAM, FOXN1, KRT8, KRT5, DLL1, DLL4, PD-L1, AIRE, HLA-DQ (class II), Tissues restricted antigens (TRAs) including but not limited to INS, IA2, GAD1, Myelin Basic Protein and Thyroglobulin; and a plurality of HPCs. In certain embodiments, compositions disclosed herein include, but are not limited to a mixture of a plurality of mammalian thymic cells including thymic stromal cells and thymic epithelial cells and HPCs having one or more specified HPC markers including CD34 and CD45. HPCs can be obtained by isolated CD34+ cells from patients or cord blood or by differentiated of human pluripotent stem cells and/or stem cells into HPCs. In some embodiments, compositions disclosed herein include a plurality of mammalian thymic encompassing mammalian HPCs expressing one or more of CD34 and CD45, CD5, CD7, CD43, CD3, TCRa/b, CD4, CD8, PD1, NCAM+, CD34+, VECAD+, and CD90+ or all markers. In accordance with these embodiments, incubation of the above referenced mixtures of cells generates AIRE positive thymic cells for therapeutic use and further study.

In some embodiments, compositions disclosed herein can include a mixture of a plurality of mammalian thymic cells including thymic stromal cells and thymic epithelial cells and a plurality of mesenchymal cells. In certain embodiments, compositions herein can further have a plurality of mesenchymal cells wherein the mesenchymal cells express PDGFRα. In other embodiments, compositions disclosed herein can further include a plurality of mesenchymal cells expressing at least one of the following markers including, but not limited to, CD105, CD90, CD73, VIM, PDGFRb, and TE-7.

In some embodiments, compositions herein include a mixture of a plurality of mammalian thymic cells including thymic stromal cells and thymic epithelial cells and a plurality of mesenchymal cells and further include HPCs contemplated herein. In certain embodiments, compositions disclosed herein can have a plurality of mammalian thymic cells including thymic stromal cells and thymic epithelial cells expressing one or more of EPCAM, CD205 and FOXN1 a plurality of mesenchymal cells PDGRFα. Mesenchymal cells can be obtained by isolation from human tissue samples, including human thymi, or by differentiation of human pluripotent stem cells and/or stem cells into mesenchymal cells, including splanchnic mesenchyme; and further include HPCs for rapid generation of targeted thymus cells of use for therapeutic applications.

In some embodiments, compositions including a plurality of HPCs can further include HPCs having one or more HPCs markers such as CD34+ HPC cells of CD34 and CD45 positive cells. In some embodiments, compositions herein can further have a plurality of HPCs expressing one or more of CD34 and CD45, CD5, CD7, CD43, CD3, TCRa/b, CD4, CD8, and PD1.

In some embodiments, compositions including mixtures of cells indicated in the paragraphs above can further contain thymus cells expressing one or more of CD205, EPCAM, FOXN1, KRT8, KRT8, DLL1, DLL4, PD-L1, AIRE, HLA-DQ (class II), Tissues restricted antigens (TRAs) including but not limited to INS, IA2, GAD1, Myelin Basic Protein and Thyroglobulin, PDGFRα, CD34 and CD45, CD5, CD7, CD43, CD3, TCRa/b, CD4, CD8, PD1, CD125 NCAM+, CD34+, VECAD+, and CD90+, CD105, CD90, CD73, VIM, PDGFRb, TE-7. over the course of differentiation. In some embodiments, T cell differentiating populations of cells disclosed herein can express one or more CD205, EPCAM, FOXN1, KRT8, KRT8, DLL1, DLL4, PD-L1, AIRE, HLA-DQ (class II), Tissues restricted antigens (TRAs) including but not limited to INS, IA2, GAD1, Myelin Basic Protein and Thyroglobulin, PDGFRα, CD34 and CD45, CD5, CD7, CD43, CD3, TCRa/b, CD4, CD8, PD1, CD125 NCAM+, CD34+, VECAD+, and CD90+, CD105, CD90, CD73, VIM, PDGFRb, TE-7 after about 0 days to about 30 days during course of differentiation.

In certain embodiments, compositions containing mixtures of cells disclosed herein as indicated at least in the paragraphs above can be incubated for a period of time in order to generate thymus cells expressing at least the cell marker AIRE protein after about 1 hour to about 1 day or less than 20 days of accelerated and improved differentiation. In other embodiments, compositions containing mixtures of cells disclosed herein as indicated at least in paragraph [0009] above can be incubated for a period of time in order to generate CD4+/CD8+ T cells after about 1 hour of incubation to about 20 days of accelerated and improved differentiation. In certain embodiments, a combination of mammalian thymic cells including thymic stromal cells and thymic epithelial cells, a plurality of mesenchymal cells and HPCs provide for a more rapid differentiation and production of desirable cells containing these markers.

In some embodiments, thymus cells and thymic tissues generated by compositions and methods disclosed herein can be harvested from a differentiated culture (after a pre-determined differentiation period) to be used immediately, after a day or several days in a therapeutic application. In some embodiments, compositions herein can be harvested from the culture and optionally purified immediately before use in a therapeutic application. In other embodiments, thymic cells and/or thymic tissues generated by compositions and methods disclosed herein can be harvested and stored at about −80° C. to about 4° C. for a period of time before use in a therapeutic setting. In some embodiments, thymic cells and/or thymic tissues generated by compositions and methods disclosed herein can be harvested and stored at about −80° C. to about 4° C. (refrigeration or on ice or dry ice) for about a day to about a year before use in a therapeutic application. In some embodiments, thymic cells and/or thymic tissues generated by compositions and methods disclosed herein can be harvested and subjected to at least one freeze-thaw cycle before use in a therapeutic application. In certain embodiments, thymic cells and/or thymic tissues generated by compositions and methods disclosed herein can be harvested by methods known in the art for delivery and therapeutic use or stored at reduced temperatures for later use or cell number expansion such as culturing.

In some embodiments, the present disclosure provides compositions and methods for generating differentiated thymic epithelial cells expressing specific markers. In some embodiments, compositions and methods disclosed herein provide for improved production and differentiation into desirable T cell populations such as production of thymic epithelial cells expressing AIRE. In some embodiments, methods disclosed herein concern mixing populations in culture of a plurality of mammalian thymic cells including thymic stromal cells and thymic epithelial cells, a plurality of mesenchymal cells and a plurality of HPCs and incubating the mixture for a period of time and generating thymic epithelial cells expressing AIRE as a marker where the mixed population is incubated for about 1 week to about 3 months. In some embodiments, compositions, and methods for generating thymic epithelial cells expressing AIRE can include incubating a population of thymic stromal cells and thymic epithelial cells disclosed herein with a plurality of mesenchymal cells disclosed herein. In other embodiments, compositions, and methods for generating thymic epithelial cells expressing AIRE can include incubating a population of a plurality of HPCs disclosed herein with a plurality of mesenchymal cells disclosed herein. In yet other embodiments, compositions and methods for generating thymic epithelial cells expressing AIRE can include incubating a population of thymic stromal cells and thymic epithelial cells, a plurality of HPCs disclosed herein and a plurality of mesenchymal cells disclosed herein for a period of time.

In some embodiments, the present disclosure provides methods for treating a subject having or suspected of having a health condition concerning the thymus or adaptive immunity. In other embodiments, compositions and methods for treating a subject having or suspected of having a health condition concerning the thymus or adaptive immunity can include delivering a population of thymic epithelial cells or thymic organoids created by methods disclosed herein to the subject. In accordance with these embodiments, cells or organoids can be harvested, prepared, and transported for administration to the subject by any means known in the art (e.g. bolus administration or infusion or transplantation).

In other embodiments, the present disclosure provides kits for storing, transporting, or practicing one or more of the compositions and methods disclosed herein. In some embodiments, kits can include one or more of cell populations or mixture of cell populations or products produced by methods disclosed herein. In other embodiments, kits disclosed herein can one or more of cell populations or mixture of cell populations or products produced by methods disclosed herein and at least one container.

In some embodiments, the present disclosure provides animal models for assessing therapies to treat a health condition or for producing an expanded population of cells such as humanized cell populations. In some embodiments, animal models can include an in vivo screening platform for one or more therapies to treat a health condition. In some embodiments, animal models herein can be in vivo screening platforms for one or more chemical-based and/or immuno-based therapies to treat a health condition. In some embodiments, animal models herein can be used for assessing therapies to treat a cancer, a thymic condition, and immune deficiency, a viral infection, and/or a bacterial infection and/or aging.

In certain embodiments, health conditions contemplated herein to be treated by cell populations and/or organoids produced herein can include, but are not limited to, a disorder of the thymus. In accordance with these embodiments, a disorder of a thymus can include, but it not limited to, an ablated, missing, malfunctioning, injured or other thymus condition. In other embodiments, thymic cells and/or thymic organoids generated herein can be patient specific or patient typed (e.g. donor screened) using patient derived or donor screened cell cultures.

Embodiments disclosed herein, compositions and methods disclosed herein concern compositions and methods for generating thymic cells and organoids able to mimic functional thymus and provide replacement T cells. For example, compositions and methods disclosed herein concern inducing differentiation of, maturing and/or educating thymocytes (positive and negative selection), including expression of genes necessary for such differentiation, education and maturation (e.g. autoimmune regulatory genes, and tissue restricted antigens), spontaneously producing medullary thymic epithelial cells, and cortical thymic epithelial cells. In some embodiments, organoids disclosed herein can spontaneously organize and develop three-dimensional structures, that include cell types that are not found in cultures using existing methods. In certain embodiments, CD4+CD8+ T cells can be generated that react to self-antigens (e.g., proteins, nucleic acids, etc. produced by the host) can be induced to undergo apoptosis by the disclosed organoids, avoiding autoimmune-related issues generated by other methods to create thymic cells and organoids known in the art.

The adaptive immune system is a critical component of the mammalian immune system, providing protection against foreign antigens. Unlike the innate immune system, the adaptive immune system is comprised of various cells that interact to mount a response against specific foreign antigens. This allows for greater (and increasing) specificity and control of the immune response, especially when compared to some innate immune responses.

The adaptive immune system is made up of two major cell types, B cells and T cells, named for where these cells develop, the bone marrow and thymus, respectively. The focus of the present disclosure is on the T cell component and the organ involved in T cell development, the thymus. The thymus is a glandular organ located in the central chest cavity and made up of a myriad of cells including parenchymal cells and stromal cells. The stromal cells include thymic epithelial cells, thymic mesenchyme, resident thymic dendritic cells, and developing thymocytes. Elaborate crosstalk between the various cell types results in the development of both the thymic and T cell compartments.

The primary role of the thymus is to develop and educate T cells. Thymic epithelial cells (TECs), which make up about two percent of the thymus, are primarily the cells that facilitate this T cell development. The thymus is divided into two distinct compartments, the cortex and the medulla, including cortical TECs and medullary TECs (cTECs and mTECs), respectively. The cTECs and mTECs each play a distinct role in the development of functional T cells, with cTECs facilitating the positive selection of developing T cells with a properly recombined and functional T cell receptor (TCR), and mTECs facilitating the process of negative selection of auto-reactive T cells. Together, these two TEC subtypes establish a T cell repertoire able to recognize and respond to foreign antigens that are presented by self-specific human leukocyte antigens (HLAs), while being tolerant to self-peptides. Aberrant negative selection in the thymus can lead to downstream problems with autoimmunity.

The adaptive immune system plays a pivotal role in processes such as cancer, organ transplantation, and autoimmunity, and the like. Previous studies have focused on T cell development and function have been limited to mouse or xenogeneic models. While these studies have elucidated many key mechanisms and aspects of human and murine thymus development and function, the field currently lacks an adequate and patient-specific model of the human thymus and lacks the ability to generate differentiated T cells and organoids generated by compositions and methods disclosed herein.

Some embodiments disclosed herein concern a model system and novel approaches allowing the generation and study of a developing, functional thymus including a human thymus, in the absence of non-human tissues and cells which has not been successful until the instant disclosure. Certain embodiments concern systems that are of isogenic nature, e.g. all cell components combined to create STOCs are directly differentiated from the same human pluripotent stem cell lines (hPSCs) but other HPCs are contemplated of use herein. In some embodiments, these hPSCs are patient-derived or patient specific for use in generating patient specific therapies disclosed herein. In other embodiments, compositions, methods and systems disclosed herein can be useful for research and cell-therapy purposes.

Certain embodiments concern in vitro thymus models that are useful in researching the human immune system, create humanized animal models, as well as the development of novel therapies, for example cell and tissue-based replacement therapies. Compositions and method disclosed herein provide advances from using current in vivo models for human thymus development and function which rely on the use of human fetal thymus and liver tissue transplanted into (NOD)-scid IL2rγ(null) (NSG) mice, or other immunodeficient mouse models. While these models provide a system for the study of viral pathologies and the immune response to various viruses, these models do not provide the ability to closely interrogate the mechanisms of human thymus development and function and do not provide a model for studying other health conditions that affect immunity and further are typically allogeneic, because the fetal thymus tissue and the HSCs are derived from different sources. In certain embodiments, models provides herein can be used to study and treat disorders including, but not limited to cancer, organ and cellular transplantation and autoimmunity. In certain embodiments, compositions, methods, and systems are disclosed providing a platform model for interrogating mechanisms of thymus development and function. In certain embodiments, compositions, methods, and systems disclosed herein provide a platform model for interrogating mechanisms of human thymus development and function

Recent work has shown that human embryonic mesoderm progenitors (hEMPs) may be generated from human pluripotent stem cells (hPSCs). When co-cultured with MS5-DLL4 cells as ATOS, hEMPs are able to develop into functional T cells. Additional, recent work has shown the generation of HSC/T-cell progenitors by direct differentiation. Unfortunately, these models still rely largely on the use of murine cell lines, making the system xenogeneic. This system also does not allow for negative selection, as the human T cell progenitors are educated by murine cells that do not express MHC class II or the critical gene, autoimmune regulator (AIRE) gene. Compositions and methods disclosed herein surprisingly circumvent these issues and overcome issues with negative selection.

### Thymic Cells (TCs)

Thymic cells, as referenced herein, can refer to epithelial and stromal cells of the thymus, but not thymocytes or developing T-cells. Thymic epithelial cells (TECs) develop from thymic epithelial progenitors TEP. However, TEPs require interactions with T cell progenitors, for example isolated CD34+ cells from patients or stem cell derived hEMPs or CD34+ cells, in order to achieve full maturity as functional thymic epithelial cells (TECs), capable of supporting functional T cell development. In some embodiments, compositions, methods, and systems disclosed herein provide for novel mixtures of cells and tissues for differentiation of patient-specific TEPs into functional, patient-specific TECs in vitro and in vivo. It was demonstrated herein that transplantation into nude, athymic, mice supports that the disclosed TEPs further differentiated into functional mature TEC cells.

In accordance with some embodiments, the instantly described TEPs can be derived from various sources, including human embryonic stem cells (hESCs), induced pluripotent stem cells, (iPSCs), referred to as human pluripotent stem cells (hPSCs). In one embodiment, the disclosed TEPs can be derived from hPSCs. In other embodiments, TEPs derived from hPSCs can provide for generating patient-specific thymic cells and tissues. In one embodiment, TEPs are prepared for example, by the method describe in PCT/US2020/30130 (incorporated herein by reference in its entirety).

In certain embodiments, compositions and methods disclosed herein concern generating various TECs and methods for making and using the TECs. In accordance with these embodiments, TECs of the instant inventions can be derived from various sources, including one or more of stem cells, for example embryonic stem cells, pluripotent stem cells, induced pluripotent stem cells available from any source. In other embodiments, HPCs can be used to generate thymic epithelial cells (TEPs). STOC-derived TECs of embodiments disclosed herein can also express various genes required for negative selection of developing T-cells. In accordance with these embodiments, these genes and markers include AIRE and can further include one or more additional downstream targets, TRAs, or tissue restricted antigens (e.g. INS, IA2, GAD1, Myelin Basic Protein and Thyroglobulin). In other embodiments, other thymic epithelial markers expressed by the disclosed TECs can include one or more of FOXN1, KRT5, KRT8, HLA-DR (MHC-II), and DLL4. In certain embodiments, expression of one or more thymic epithelial markers in a population of TECs can lead to an increase beginning when STOCs are formed until about 6 weeks, for example by greater than 2-fold, 10-fold, or 100-fold, relative to day 0 and other methods known in the art. In some embodiments, STOCs can be formed from about 2 weeks to about 4 weeks using mixtures of cells disclosed herein and provide superior thymic cell populations, for example in about 10 days, 12 days, 14 days, 16 days, 18 days, 20 days, 22 days, 24 days, 26 days, 28 days, 30 days, or 32 days, and less than about 34 days, 32 days, 30 days, 28 days, 26 days, 24 days, 22 days, 20 days, 18 days, 16 days, 14 days, or 12 days compared to other methods.

### Hematopoietic Cells

In some embodiments, hematopoietic cells (HPCs) can be used in mixtures of cells and cell tissues for creating thymic co-cultures and organoids. In certain embodiments, HPCs of use herein express one or more of CD34+, CD45+, and CD43+. In some embodiments, HPCs can be hematopoietic progenitors, for example hemogenic cells, expressing one or more of EPCAMneg/low, NCAM+, CD34+, VECAD+, and CD90+. In some embodiments, hematopoietic cells can be obtained or derived from various sources, including mesodermal cells, hemogenic cells, hematopoietic stem cells (HSCs), cord blood, bone marrow, peripheral blood from an activated donor, dental tissues, skin tissues or any other source. In certain embodiments, HPCs contemplated of use herein can be derived from induced pluripotent stem cells (iPSCs) for example human iPSCs, and/or human embryonic mesodermal progenitor cells (hEMPs) and/or hematopoietic organoids. In some embodiments, cells can be sorted using marker indicators and cell sorting before co-culture. In some embodiments, cells can be sorted using surface markers including, one or more of EPCAMneg/low, NCAM+, CD34+, VECAD+, RAG1+, CD31+ and CD90+. Hematopoiesis in human stem cell culture can differentiate through hEMPs that can give rise to functional T cells. hEMPs can be generated by exposing cells to one or more of ActivinA, BMP4, VEGF and FGF stimulation. In some cases, ROCK inhibitor can be provided.

### Thymic Organoid

In some embodiments, methods for producing thymic organoids are disclosed for in vivo and in vitro study and/or use in therapeutic treatments. In certain embodiments, the disclosed thymic organoids can include hematopoietic or hemogenic cells and thymic cells and mesenchymal cells. In accordance with these embodiments, hematopoietic cells, thymic cells and mesenchymal cells can be allogeneic, autologous or isogenic. In other embodiments, organoids produced by compositions and methods disclosed herein can have a three-dimensional structure that is similar or the same as structures found in a native human thymus. In other embodiments, HPCs, thymic cells and mesenchymal cells can be co-cultured for more than about 1 week and less than about 16 weeks, for example more than about 1, 2, 3, 4, 5, 6, 7, 8, 9, 10, 11, 12, 13, 14, or 15 weeks, and less than about 20, 19, 18, 17, 16, 15, 14, 13, 12, 11, 10, 9, 8, 7, 6, 5, 4, 3, and 2 weeks in order to form the organoid and produce differentiated thymus cells with desired features disclosed herein. In some embodiments, mesenchymal cells are not added to thymic organoids and mesenchymal cells will spontaneously form in thymic organoids and can be readily detected after about 2 weeks and up to about 3 months. When mesenchymal cells are not added, further differentiation of thymic cells is delayed and thymic cells quality is impaired, e.g. AIRE protein cannot be detected. In some embodiments, HPCs are not added to thymic organoids but, contrary to current literature, thymic cell differentiation appears comparable to thymic organoids containing, thymic cells, HPCs and mesenchymal cells as discovered and disclosed herein.

In other embodiments, organoids generated by methods disclosed herein can include cells that spontaneously organize and form structures and environments that mimic a functional human thymus. In some embodiments, organoids created by methods disclosed herein can spontaneously organize in the absence of mesenchyme but take about 5 weeks to about 10 weeks to form structures and environments that mimic a functional human thymus. In some embodiments, organoids created by methods disclosed herein can spontaneously organize in the absence of mesenchyme but take about 5 weeks to about 10 weeks to form structures and environments that mimic a functional human thymus and where the cells do not express AIRE protein which indicate that they are of inferior quality to those formed in the presence of mesenchyme. In some embodiments, organoids created by methods disclosed herein can spontaneously organize in the absence of mesenchyme but take about 5 weeks to about 10 weeks to form structures and environments that mimic a functional human thymus and include less than about 10% T cells that are CD4+/CD8+ in the absence of mesenchyme where levels of 20% and more T cells that are CD4+/CD8+ have been demonstrated. In accordance with these embodiments, the addition of mesenchyme to mixtures for producing organoids disclosed herein reduced production time and generates superior quality structures with increased populations of T cells that are CD4+/CD8+ and T cells that express AIRE protein.

In other embodiments, compositions and methods disclosed herein for generating organoids can include cells that organize and form structures and environments that mimic a functional human thymus in the presence of mesenchyme. In some embodiments, compositions and methods disclosed herein for generating organoids can include cells that organize and form structures and environments that mimic a functional human thymus in the presence of mesenchyme that can take about 1 week to about 3 months to form structures and environments that mimic a functional human thymus, much faster than without mesenchyme. In other embodiments, compositions and methods disclosed herein for generating organoids can include cells that organize and form structures and environments that mimic a functional human thymus in the presence of mesenchyme can take about 1 week to about 3 months to form structures and environments that mimic a functional human thymus wherein the structures express AIRE protein. In some embodiments, compositions and methods disclosed herein for generating organoids can include cells that organize and form structures and environments that mimic a functional human thymus in the presence of mesenchyme can take about 2 weeks to about 5 weeks to form structures and environments that mimic a functional human thymus wherein the structures include more that about 10% to about 100% (e.g., more than about 10%, 20%, 30%, 40%, 50%, 60%, 70%, 80%, 90%, 95%, 99%, 100%) T cells that are CD4+/CD8+. In other embodiments, single positive cells can be formed, both CD8 and CD4; preferences to CD8. In the early stages of mixtures of the 3 cell/tissue types (e.g. thymus, HPCs and mesenchyme), HPCs can provide progenitors and this leads to production of double positive cells CD4/8 which can further develop into SP cells. At early times, double positive cell are the majority, but as HPCs exhaust this will shift towards a more and more single positive phenotype with less and less and eventually no double positive developing T cells.

In some embodiments, organoids generated by compositions and methods disclosed herein can include parenchymal cells and stromal cells, for example thymic epithelial cells, thymic mesenchyme, thymic dendritic cells, thymocytes, and other cells. In certain embodiments, organoids generated by methods disclosed herein support signaling/crosstalk between the various cell types to aid in the development of both thymic cells and immune cells.

In certain embodiments, organoids generated by methods disclosed herein can include one or more other types of cells. In some embodiments, the other types of cells can be one or more of thymic mesenchymal cells, endothelial cells, antigen presenting cells, macrophages, and dendritic cells of use to treat a variety of health conditions and for use in creating specific animal models such as humanized animal models.

In certain embodiments, organoids generated by methods disclosed herein can include one or more mature or maturing hematopoietic cells that express one or more markers expressed by developed or developing T-cells. In some embodiments, a marker can include one or more of, CD3, TCRa/b, TCRd/g, CD5, CD7, CD4, CD8, CD45, CD125, PD1 and FOXP3. In other embodiments, T-cell markers can be upregulated and can reach or be near a maximal level in as few as about 2 weeks or less compared to when the cells of the organoid are first co-cultured or in the absence of mesenchyme. In other embodiments, maximal expression of markers disclosed herein and desired can be from about 2 to 6 weeks, for example about 15 days, 20 days, 25 days, 30 days, 35 days, or 40 days, or less than about 45 days, 40 days, 35 days, 30 days, 25 days, or 20 days. In many embodiments, upregulation may be 2-fold, 10-fold, 100-fold, or greater.

In other embodiments, organoids generated by compositions and methods disclosed herein can include one or more thymic epithelial cells expressing one or more markers indicative of mature or maturing thymic epithelial cells. In accordance with these embodiments, these markers can include one or more markers including, but not limited to, CD205, EpCAM, AIRE, FOXN1, KRT5, KRT8, HLA-DR (MHC-II), PD-L1, DLL1, DLL4, INS, IA2, GAD1, Myelin Basic Protein and Thyroglobulin. In other embodiments, the disclosed marker may be upregulated from about 2 weeks to about 6 weeks, for example at 3 weeks of culture compared to when the cells of the organoid are first co-cultured. In certain embodiments, upregulation can be about 2-fold, 10-fold, 100-fold, or greater compared to organoids and T cells not produced by the current methods. In some embodiments, thymic cells and tissues produced by compositions and methods disclosed herein can be equal to or superior to healthy naturally occurring thymic tissues and cells. In other embodiments, other thymic epithelial markers expressed using compositions and methods disclosed herein can include, for example CXCL12 and CCL25, which can be upregulated by about 5-fold or more.

### Use of Thymic Cells and Thymic Organoids

In other embodiments, compositions, systems and methods disclosed herein include creating and use of novel stem cell derived thymic organoid cultures (STOCs). In accordance with these embodiments, STOCS created herein can be used in creating a functional equivalent of a mammalian thymus. The disclosed STOCs can be generated allogenically or isogenically. In some embodiments, the STOCs are generated allogenically using variously sourced HSCs, which may include but are not restricted to, cord blood and CD34+ HSC cells isolated from activated adult patient blood, (e.g. by treatment with human granulocyte-colony stimulating factor with or without cyclophosphamide). In other embodiments, mesenchymal cells from primary human tissues, including thymic tissues can be added to create STOCs. In other embodiments, STOCs disclosed herein can be generated isogenically. In accordance with these embodiments, HSC progenitors and mesenchymal cells are generated from the same or a similar cell line to that used to generate the TEPs.

In other embodiments, it is noted that generation of organoid and T cells of the present disclosure is the first demonstration of AIRE and TRAs expression in stem cell derived thymic cells. In accordance with these embodiments, expression of AIRE and TRAs is a feature of a fully functional thymus which is important to negative selection of T cells (e.g. to reduce autoimmunity). Independent of the HSC source, the present disclosure allows for developing T cells, at distinct stages of education, to be identified. Cells identified, using the currently disclosed compositions, cells, methods, protocols and systems can include one or more of positively selected CD3+ cells, double positive CD4 and CD8 cells, and single positive T cells. Transplantation of the presently disclosed STOCs, into preclinical animal models, results in the appearance of circulating T cells. Grafts created by the disclosed compositions, cells, methods, and systems exhibit KRT8 and KRT5 marker expression for cTEC and mTEC, respectively. Further, the disclosed grafts also possess structures that are similar to Hassall's corpuscle, which is a structural characteristic of a functional thymus.

In other embodiments, compositions, modified cells, methods, protocols, and systems are disclosed for the generation of a functional human thymus under cell culture conditions. In certain embodiments, the disclosed functional thymus supports positive and negative education of thymocytes. In certain embodiments, the disclosed functional human thymus can be patient specific. Moreover, the presently disclosed thymi can be achieved using various approaches or procedures for preparing cell populations. In other embodiments, the disclosed thymic co-cultures exhibit various features useful in education of developing T-cells. Surprisingly, in contrast to other methods and cultures, the disclosed thymic structures created herein support negative selection of T-cells. Negative selection permits educating developing T cells, for example in an isogenic manner. In certain embodiments, hPSCs can be used to generate the co-culture/thymic structures. In some embodiments, these generated thymic structures allow transplanted cells and tissues to remain functional upon transplantation into preclinical animal models or animal or human patients.

### Isogenic Therapy Study

In some embodiments, development of the presently disclosed in vitro-generated human thymus can be useful for various purposes. In certain embodiments, the disclosed cells and tissues can be useful in developing novel cell therapies, including patient-specific cell therapies. The disclosed cells and tissues can also be useful in modeling various diseases and conditions, for example immunodeficiency, cancer, and autoimmune diseases. In certain embodiments, these models can be patient-specific, allowing development and/or testing of various therapies that can be designed and tested for specific treatment of an individual patient, personalized analysis and treatment.

In accordance with these embodiments, health conditions such as congenital diseases, such as DiGeorge syndrome and loss of function FOXN1 mutations can result in the absence of a functional thymus and severe immunodeficiency in humans where thymic structures and organoids disclosed herein can be used to treat such a condition. Current treatments for such congenital disorders typically involve the transplantation of fetal or neonatal thymus tissue into the affected individuals (usually the thigh). However, such treatments rely on access to these tissues, which can be very limited. Embodiments disclosed herein allow for generation of patient-specific thymic organoids that can be used in overcoming the lack of tissue and/or treating these diseases. Indeed the presently disclosed compositions, cells, methods, protocols, and systems can provide a source of large quantities of cells and or tissue for replacement therapy in shorter periods of time.

It is understood that because hPSCs are amenable to genome editing, the instantly disclosed compositions, cells, methods, and systems can be used in rectifying one or more genetic mutations, for example mutations that result in the initial loss of thymic tissue. DiGeorge syndrome patients commonly experience complete or partial loss of TBX1, a factor involved in the development of the thymus. In some embodiments, compositions and methods for generating thymic tissues disclosed herein can provide for cell replacement therapy that can include correcting such a genetic mutation in the hPSCs prior to differentiation of these cells into thymic epithelial cells. Additionally, such an approach can be useful in treating various autoimmune diseases, such as type 1 diabetes. In accordance with these embodiments, the disclosed compositions, cells, methods, and systems can be useful in re-inducing tolerance by (re-) educating existing patient T-cells and/or providing T regulatory cells in thymic cell systems. In some embodiments, the T-cells or T-regulatory cells can be gene edited or altered to facilitate desired gene expression, for example to express auto-antigens and/or specific TCRs and/or chimeric antigen receptor. Such approaches may be useful to provide tolerance in autoimmune settings e.g. autoimmune diabetes directed at insulin producing beta-cells or in an allogenic setting, e.g. solid organ transplantations. Tolerance induction to allogeneic tissues has been demonstrated by using mouse models which would be similar to the use of technologies disclosed herein. For example, these inventions are beneficial in the clinical setting in inducing tolerance during allogeneic organ transplantations, eliminating the need for immunosuppressant therapy by using personalize approach to generating thymic cells and organoids disclosed herein.

### Longevity—Thymic Augmentation/Replacement

In other embodiments, compositions, cells, methods, and systems disclosed herein can be used to enhance immune function and/or longevity in a subject. The thymus undergoes age related involution, characterized by decrease thymic output of naïve T cells, and a decreased ability to respond to new immunological assaults, including cancer. Studies in mice have shown that transplanting a young thymus into an old mouse can significantly increase the life span of the recipient mice. In certain embodiments, compositions, cells, methods, and systems can be used for providing an autogenic thymus to a subject experiencing involution, due to age, immune suppression (e.g. transplant patients), exposure to anti-thymic compounds (for example chemotherapeutic compounds), and various diseases and disorders. This can result in increasing the span and/or quality of life for subjects treated with the disclosed therapies. The disclosed thymic augmentation can be used in a subject having lymphopenia, for example lymphopenia that is the result of chemotherapeutic treatment for cancer. In other embodiments, the disclosed thymic augmentation can be used to treat a subject preparing for hematopoietic stem cell (HSC) transplant. In some embodiments, patient-specific thymic organoids disclosed herein can be used in the reconstitution of the immune system in such patients, aiding in recovery.

Isogenic, patient-specific thymic organoids of embodiments of the instant application can be used in therapies as well as patient-specific disease modeling. Patient-specific disease modeling can be accomplished in vitro and in vivo using compositions and methods disclosed herein. The presently disclosed thymic organoids can be subjected to genetic manipulation, including genome editing. This type of modification can be used in creating cell lines, organoids, etc. for investigation of various diseases, disorders, and conditions, for example genetic defects, such as downs syndrome. The disclosed organoids can allow for the study of the effects of such conditions on thymus development in-vitro. This can allow for identification of various molecular mechanisms involved in thymus development, as well as the function of molecular mechanisms and pathways in disease, potentially leading to identification of treatment modalities.

In other embodiments, isogenic thymic organoids generated by compositions and methods disclosed herein can be useful in developing humanized mouse models that more closely resemble and function as human immune systems, which can provide for improvements in the study of thymic disease, function, and development. In other embodiments, thymic organoids disclosed herein can facilitate investigation of various pathologies, such as cancer, viral infection, autoimmune and other. In one embodiment, the disclosed thymic organoids can aid in investigation of immune/tumor interaction in a variety of cancers, and allow for development of enhanced therapies for treatment of these conditions using a model system or a personalized model system of organoids disclosed herein.

In some embodiments, thymic organoids developed by compositions and methods disclosed herein can be used for tailoring treatment protocols for specific patients. In accordance with these embodiments, peripheral blood mononuclear cells (PBMCs) can be isolated from a patient having a disease or condition. The PBMCs can then be reprogramed to create patient-specific iPSCs. The patient-specific iPSCs can then be transplanted into immunodeficient mice as isogenic, patient-specific iPSC-derived thymic organoids. Tissue from the patient, for example tumor tissue, can be isolated and transplanted into the same mice housing a replica of that patient's thymus. This system provides a model system for interrogating a patient's immune response to a cancer. In some embodiments, this system can allow for the isolation (e.g. cloning, expansion, etc.) of antigen specific T cells to treat the tumor in the patient by personalized methods.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of embodiments of the inventions.

### Example 1

In one exemplary method, iPSC cells were used as a source for deriving TEPs, which were analyzed for differentiation into functional TECs in vitro. Stem cell derived thymic organoid co-cultures (STOCs) were established. FIG. 1 depicts a schematic providing an overview of the exemplary method. Briefly, iPSCs were used to produce TEPs. CD34+ HSCs cells were isolated from human umbilical cord blood. TEPs were then aggregated with the CD34+ HSCs at the air-liquid interface (FIG. 2A). qPCR analysis was used to investigate the STOCs at various time points. FIGS. 2B-2I show exemplary gene expression analyses of TEP markers FOXN1, AIRE, KRT5, KRT8, HLA-DR, DDLL4, CXCL12, and CCL25. Various thymic markers, FOXN1 (FIG. 2B), KRT5 (FIG. 2G), KRT8 (FIG. 2F), HLA-DR (FIG. 2D), DLL4 (FIG. 2E), were upregulated after 3 weeks. These results indicated that the iPSC derived TEPs were maturating into TECs in vitro. AIRE transcripts were upregulated in the STOCs at 3 weeks (FIG. 2C). This is in contrast to observations in vivo using the xenogenic nude model. Accordingly, in vitro maturation induced mature TEC markers: FOXN1 and AIRE (key Thymic transcription factors); KRT8 and KRT5 (Markers of TEC specific epithelium); and MHC-II and DLL4 (Necessary for T cell development). CXCL12 (FIG. 2H) and CCL25 (FIG. 2I) were not seen to be upregulated in STOCs. Without wishing to be limited by theory, this may be because HSCs do not need to be attracted into the thymic microenvironment in this system.

Gene expression analysis of tissue restricted antigens (TRAs) markers [insulin (INS; FIG. 2J), islet antigen 2 (IA2; FIG. 2K), glutamate decarboxylase 1 (GAD1; FIG. 2L), myelin basic protein (FIG. 2M), and thyroglobulin (FIG. 2N)] showed expression in STOCs after 3 weeks, but not at the start of the experiment. These data confirmed functional AIRE expression. Note expression of TRAs typically expressed from cells of different germ layers, for example ectodermally (myelin basic protein) and endodermally (INS, IA2, thyroglobulin and GAD1) expressed TRAs

STOC sections were analyzed at 3 weeks, by IF, for various epithelial and mesenchymal markers, for example EPCAM and PDGFRα respectively. This analysis revealed thymic epithelial structures surrounded by mesenchymal cells (FIG. 2O). Co-staining of EPCAM+ cells with a pan-Keratin antibody showed co-expression in almost all epithelial cells (FIG. 2P). To identify m and cTECs directly, sections were stained for KRT5 and KRT8, respectively. The majority of all TECs were single positive for KRT8, suggesting a predominant cTEC phenotype of STOCs. In addition, many KRT5/8 double positive cells, indicative of developing TECs were present while only few single positive KRT5 mTECs are found (FIG. 2Q). Further IF analysis of STOC sections for T cell markers CD4 and CD8 showed double positive, (DP) developing T cells located near pan-KRT positive thymic structures (FIG. 2R). Quantification of emergence of progenitor T cells, marked by CD5 and CD7, was performed by flow cytometric based analysis for progenitor T cell markers (pre-/pro-T cells), CD5 and CD7, and T cell markers, CD4 and CD8, after 2, 3, and 6 weeks of culture as STOCs. This analysis identified low levels of proT cells at week 2 followed by a substantial increase at week 3 that was sustained throughout the culture period assayed (FIGS. 2S-2T).

FIGS. 3A-3B show activation of carrier T cells in allogenic STOCs by a representative flow cytometry analysis of activation marker CD25 at weeks 2, 3, and 6 of STOCs and quantification of flow analysis (n=2, 1 iPSC line) FIG. 3B shows percentage of CD25 as a function of time.

FIGS. 4A-4L show generating isogenic STOCS which exhibited functional thymus differentiation and facilitated T cell development in vitro. In brief, FIG. 4A shows a schematic outline of one embodiment for development of the disclosed cultures. FIGS. 4B-4G show qPCR analysis of isogenic STOCs depicting upregulation of TEC markers. Note induction of AIRE expression (FIG. 4C). FIGS. 41I-4J show IF staining of mesenchymal, epithelial and TEC markers depicting development of mTECs in isogenic STOCs. Note spontaneous emergence of mesenchymal cells critical in supporting TEC development and function. FIGS. 4K-4L show flow based analysis which demonstrated generation of CD5+/CD7+ pro/pre T cells, CD3+/TCRa/b+, CD4+/CD8+ double positive and either CD4+ or CD8+ single positive developing T-cells.

Quantitative flow analysis of CD4 and CD8 revealed developing, double positive T cell starting at week 2. These cells were not present in isolated CD34+ HSPCs at the start of STOC culture. Large populations of single positive CD4+ T cells and CD8+ T cells were also observed at weeks 2 and 3. Isolated CD34+ HSCs contained a small population of contaminating CD3 T cells that get activated and presumably expand considerably in the allogenic STOCs (FIG. 4K). This contaminating population prevented confirmation of de novo generation of single positive T cells in this system. STOCs were observed to decline after approximately 3 weeks in culture, resulting in loss of thymic marker expression.

These results were the first demonstration of generation of functional human TECs from iPSC using a novel STOC culture system in vitro. TECs within STOCs expressed various genes involved in negative selection of T-cells, and the TECs facilitated development of pro-T cells and double and single positive T cells from allogenic human cord blood CD34.

### Example 2

In another exemplary method, humanized mice are generated using STOC technology. FIG. 5A provides a schematic of the exemplary approach involving the use of isogenic hEMPs as the source for T cells. In brief, STOCs were transplanted subcutaneously into NSG mice that lacked an endogenous immune system. Human T-cells were detected in the blood of graft recipient mice while absent in control mice by flowcytometric quantification indicating humanization within only 10 days. Extraction of graft tissue 10 days post transplantation followed by staining for T cell and thymic marker proteins showed T cells in close proximity to thymic structures (FIG. 5A). Presence of structures resembling Hassall c corpuscles were found within grafts (FIG. 5A). Flow based analysis 10 days post engraftment showed CD45/CD3 double positive human T cells in the blood of graft bearing mice (FIG. 5B).

### Example 3

In another exemplary method, hPSC were differentiated using methods herein and assessed for EPCAM/CD205, a marker for human thymic cells. FAC sorting of thymic cells using EPCAM+/CD205+ showed enrichment of the thymic markers (FIGS. 6A-6C). Gene expression analysis was performed for unsorted cells and cells sorted for EpCAM/CD205+ expression. EpCAM/CD205+ enriched cells showed higher FOXN1, AIRE, K8, and DLL4 expression compared to unsorted cells (FIGS. 6D-6E). These results demonstrated the validity and usefulness of this surface marker and combination thereof to enrich for thymic cells.

### Example 4

In another exemplary method, the effect of Activin A on inducing thymic cell generation was assed. In brief, hPSC cells were differentiated for 10 days using methods disclosed herein. Differentiation was followed by addition of BMP4 and/or Activin A and/or inhibitors of their respective pathways (FIGS. 7A-7B). Flow based quantification of cultures from different treatment conditions demonstrated increased generation of thymic cells using the surface markers EPCAM/CD205 in the presence of ActivinA during the later stages of the direct differentiation process (FIG. 7C). Quantification of the percentage of EpCAM/CD205% positive cells at day 14 and 22 of differentiation is shown in FIG. 7D.

### Example 5

In another exemplary method, hEMPs were used to generate HSCs. In brief, semi confluent wells (70-80%) were treated with 5 ml TryplE for 7 min at 37° C. until dissociated. 1.5 ml mTeSR+ was added to quench trypsin, and the mixture was then transferred to a 15 ml conical tube with 5 ml PBS. A small aliquot was removed for a cell count. Cells were centrifuged at 1200 RPM for 3 min. Supernatant was removed and cell pellet was re-suspend at 1×10\hat{ }6 cells/100 μl in d(−17) media. Plates were seeded with 3×10\hat{ }6 cells in 3 ml of d(−17) media (FIG. 8B) into matrigel coated 6-well plates approximately 3×10\hat{ }6 cells per 6 well.

Plates were incubated under standard conditions. Media containing ActivinA, BMP4, VEGF and FGF stimulation was changed daily. At 3.5 days, cells were harvested for sorting. Wells were washed 3 times with PBS, then 1 ml of accumax/well was added and the plates were incubated for 9 min at 37° C. 2 ml mTeSR was added per well to quench. The solution was transferred to a 5 ml FACs tube with a strainer, counted, then centrifuged for 3 min at 1500 RPM. The cell pellet was resuspended in 500 flow cytometric buffer (PBS with 0.2% fetal bovine serum and 2 mM EDTA), and incubated with anti-EpCAM and anti-NCAM (1:50) for 25 min on ice. Cells were washed with 2.5 ml FC buffer, and re-suspend at 3×10\hat{ }6 cells/ml. 1 drop of 7-AAD was added to 5 ml TV for 2 min at room temperature in the dark. The reaction was then placed on ice and taken for sorting.

For STOCs, sorted hEMPs were combined with TEPs in 1:20 TEP:hEMP ratio (1/6 well TEPs with 50k hEMPs). Co-cultures were incubated for two weeks in the media shown in FIG. 8A. Media was changed every 2 days. At 2 weeks (d14) media was changed to artificial thymic organoid (ATO) media (FIG. 8C).

### Example 6

In another exemplary method, differentiation of iPSCs to splanchnic mesoderm was performed. FIG. 9A provides a schematic outlining the differentiation approach. In brief, 80-90% confluent iPSCs were washed with 1 ml of PBS and dissociated into single-cells with 1 ml of TrypLE/6-well for 7 min at 37° C. and quenched with 1.5 ml mTeSR+. Cell suspension was transferred to a 15 ml conical with PBS so that total volume was 10-15 ml. 600,000 iPSCs were seeded per well in a Matrigel coated 24-well plate in 500 μl mTeSR+ with Rock Inhibitor (10 μM, 1:1000). On day 0, cells were washed once with koDMEM/F12 and differentiation was induced by adding d0 media (see Table 1 for schedule and Table 2 for factors used). A basal media composed of Advanced DMEM/F12, N21, B27, 15 mM HEPES, 2 mM L-glutamine, penicillin-streptomycin was used for this and all subsequent differentiations. In addition, these buffers can be replaced with other buffers including, 50×N21 and 50×B27. In certain examples, N21 is a protein supplement needed for these compositions for more optimal outcome. In other exemplary methods, growth factors and small molecules disclosed herein can be used to obtain high percent mesenchyme.

TABLE 1

| | Day | | | | |
|---|---|---|---|---|---|
| | 0 | 1 | 2 | 3 | 4 |
| Factors | A, B, C, | A83, B, | A83, B. | A83, B. | A83, B. |
| | F, P | C59 | C59, F, R | C59, F, R | C59, F, R |
| Volume | 300 | 350 | 450 | 600 | 750 |

TABLE 2

| Factor | Concentration | Day |
|---|---|---|
| A = ActA | 30 ng/ml | |
| A83-A8301 | 1 uM | |
| B = BMP4 | 40 ng/ml | d0 |
| B = BMP4 | 30 ng/ml | d1+ |
| C = Chir | 6 uM | |
| C59 | 1 uM | |
| F = FGF2 | 20 ng/ml | |
| P = PIK90 | 100 nM | |
| R = TTNPB | 6 uM | |
| N21 | 1:50 | |

Three independent iPSC lines (2395 SM, CB002 SM, and CUHX09 SM) were differentiated to splanchnic mesenchyme (SM) for 4 days, dissociated and stained for PDGFRα (FIG. 9B-9D), quantification of which is shown in FIG. 9E.

### Example 7

In another exemplary method, differentiation of iPSCs to CD34/45 double positive hematopoietic stem cells (HSCs) was performed. In brief, 80-90% confluent iPSCs were washed with 1 ml of PBS and dissociated into single-cells with 1 ml of TrypLE/6-well for 7 min at 37° C. and quenched with 1.5 ml mTeSR+. Cell suspension was transferred to a 15 ml conical with PBS so that total volume was 10-15 ml. Cells were counted, pelleted at 1200 RPM for 3 minutes and resuspended in Stage I media so that the cell concentration was 1e6/1 ml. 5.5e6 cells were seeded in a single well of a suspension culture 6-well plate in 5.5 ml total volume of Stage I media. Cells were cultured under standard cell culture conditions on a rotary shaker at 100 RPM. On day 8, cell clusters were transferred to a 15 ml conical and allowed to settle for 3-5 minutes. The supernatant was removed, and 10-20 clusters were transferred to vitronectin coated membranes in 1.5 ml of Stage V media. On day 12-17, media was removed, and 1 ml of 1 mg/ml collagenase Type I (with DNaseI) was added under the membrane, and 0.5 ml on top and incubated at 37° C. for 1 hour. The membranes were then washed with 1 ml of TrypLE (with DNaseI) and transferred to a 15 ml conical, and incubated at 37° C. for 5 minutes, and triturated until clusters were dissociated. 5 ml of protein containing media was added to the dissociated clusters, and cell suspension was filtered through a 100 μm filter. Dissociated iPSC derived HSCs were spun down at 1200 RPM for 3 minutes, supernatant was removed, and cells were resuspended at 1e6/1 ml in Stage V media. The composition of Base, Stage I, Stage II, Stage III, Stage IV, Stage V, Stage VI, and Stage VII media are provided in Tables 3-10, respectively. Table 11 provides a schedule for media types, where media is changed on days with an asterix (*)

TABLE 3

| Base Media | Concentration |
|---|---|
| IMDM | |
| F12 Nutrient | |
| Mix | |
| Hybridoma Mix | 4% |
| Human Serum | 0.1% |
| Methyl cellulose | 0.1% |
| Glutamax | 1× |
| Ascorbic Acid | 50 ug/ml |
| ITSE AF | 1:1000 |
| Lipid Mixture 1 | 1× |
| P/S | 1× |
| BME | 22 uM |

TABLE 4

| Stage I | Concentration |
|---|---|
| Rock Inhibitor | 10 uM |
| CHIR | 0.5 uM |
| Activin A | 10 ng/ml |
| BMP4 | 20 ng/ml |
| SCF | 20 ng/ml |
| VEGF | 20 ng/ml |
| FGF2 | 10 ng/ml |

TABLE 5

| Stage II | Concentration |
|---|---|
| CHIR | 0.5 uM |
| Activin A | 10 ng/ml |
| BMP4 | 20 ng/ml |
| SCF | 20 ng/ml |
| VEGF | 20 ng/ml |
| FGF2 | 10 ng/ml |

TABLE 6

| Stage III | Concentration |
|---|---|
| CHIR | 3 uM |
| SB | 3 uM |
| Activin A | 10 ng/ml |
| BMP4 | 20 ng/ml |
| SCF | 20 ng/ml |
| VEGF | 20 ng/ml |
| FGF2 | 10 ng/ml |

TABLE 7

| Stage IV | Concentration |
|---|---|
| BMP4 | 20 ng/ml |
| SCF | 50 ng/ml |
| VEGF | 50 ng/ml |
| IGF-II | 20 ng/ml |
| FGF2 | 10 ng/ml |
| VEGF | 20 ng/ml |
| FGF2 | 10 ng/ml |

TABLE 8

| Stage V | Concentration |
|---|---|
| SCF | 100 ng/ml |
| VEGF | 50 ng/ml |
| FGF2 | 10 ng/ml |
| IL7 | 20 ng/ml |
| FLT3L | 10 ng/ml |

TABLE 9

| Stage VI | Concentration |
|---|---|
| SCF | 20 ng/ml |
| VEGF | 50 ng/ml |
| FGF2 | 10 ng/ml |
| IL7 | 20 ng/ml |

TABLE 10

| Stage VII | Concentration |
|---|---|
| VEGF | 50 ng/ml |
| IL7 | 20 ng/ml |

TABLE 11

| Day | 0 | 1 | 2 | 3 | 4 | 5 | 6 | 7 | 8 | 9 | 10 | 11 | 12 |
|---|---|---|---|---|---|---|---|---|---|---|---|---|---|
| Stage | I* | II* | III* | III* | IV* | IV | IV* | IV | V* | V* | V | V* | V |

FIG. 10A provides a schematic outlining the differentiation approach. FIG. 10B shows a representative image of human pluripotent stem cell clusters at the start of the differentiation protocol. Note that differentiation were performed in suspension culture based bioreactors capable of upscaling the process. FIGS. 10C-10E shows representative flow cytometric analysis of cultures at d12 for markers CD34 and CD45. Quantification of two independent differentiation experiments are also shown (FIGS. 10C-10D).

### Example 8

In another exemplary method, Stem derived Thymic Organoid Cultures (STOCs) were generated. FIG. 11 provides a schematic outlining the exemplary method. In brief, iPSCs were differentiated to TEPs, splanchnic mesoderm, or CD34/45 double positive hematopoietic stem cells (HSCs). At approximately day 21, TEPs were harvested using a 1 ml pipet, mechanically dissociated into small clumps, and transferred to a 1.5 ml Eppendorf tube. Primary (allogenic) or day 4 iPSC (isogenic) derived, mesenchymal cells and day 12-17 HSCs were added at a 1:1:20 mesenchyme:HSC:TEP ratio. Cell suspensions were spun down using a swinging-bucket centrifuge at 1200 RPM for 3 minutes. After careful removal of the supernatant, the cell slurry was adjusted to 5 μl per STOC and STOCs were plated by forming a drop at the tip of a 20 μl pipet tip on to a 0.4-μl Millicell Transwell insert, with 2-3 STOCs plated per membrane about 1 cm apart (EMD Millipore; Cat PICMORG50). Transwells were placed in a 6-well plate containing 1.5 ml of Stage V media (see Table 8). Media was changed every two-three days by removing half of the media and adding back 1 ml of Stage V media. STOCs were cultured in this manner for two-six weeks.

STOCs were harvested at the indicated times for IF and qPCR analysis. For IF analysis, STOCs were fixed on the membrane with 4% PFA for 15 minutes at room temperature. STOCs were then washed three times with PBS, and incubated in 30% sucrose at 4° C. overnight. STOCs were then cut from the membrane and embedded in optimal cutting temperature (OCT) medium and snap frozen. 10 μm sections were made from cryoblocks using a cryostat. For qPCR analysis, STOCs were harvested with 0.5 ml FACS buffer and transferred to a 1.5 ml tube. Cells were pelleted by quick spin using a table-top centrifuge, supernatant was carefully removed, cell pellet was resuspended in Qiagen buffer RLT lysis buffer, and stored at −80° C.

FIGS. 12A-12 illustrate the generation of TECs using STOCs consisting of TEPs, HSCs/T cell progenitors, and mesenchymal cells. FIG. 12A provides representative images showing STOCs consistent of TEPs, HSCs/T cell progenitors without or with increasing amounts of mesenchymal cells. FIG. 12B provides representative images showing IF analysis of protein expression KRT5 and KRT8 in STOCs formed with mesenchyme. FIG. 12C provides representative images of IF analysis of protein expression for epithelial marker EPCAM and mesenchymal marker PDGFRα in STOCs formed with mesenchyme. FIG. 12D provides representative images of flow-based analysis for thymic surface proteins EPCAM, CD104, CD205 and HLA-DR in primary thymus and STOCs formed with mesenchyme. FIG. 12E provides representative images of IF analysis of protein expression for AIRE, a critical regulator of negative selection of developing T cells in STOCs formed with mesenchyme.

FIG. 13 provides representative images of flow-based analysis demonstrating improved generation of single positive CD4 and CD8 positive T cells in STOCs. Flow based analysis showed the generation of CD5+/CD7+ pro/pre T-cells, CD3+/TCRa/b+, developing CD4+/CD8+ double positive and either CD4+ or CD8+ single positive T-cells.

FIG. 14 provides representative images of IF analysis of protein expression for epithelial marker EPCAM and mesenchymal marker PDGFRα and thymic mesenchyme specific TE-7 in TEP and STOC cultures. IF analysis showed spontaneous generation of mesenchyme in STOCs initially created without mesenchyme.

### Example 9

In another exemplary method, blood and melanoma tissue were collected from two human subjects (CUHM1008 or CUHM009). STOCs were generated from the blood of subject CUHM009 using the methods described herein. Next, from a cohort of 24 NSG mice, 12 were thymectomized and CUHM009 STOCs were implanted tissue in their kidney capsules. All mice were humanized using HSCs HLA-A matched to the STOCs. After identification of human T cells in the peripheral blood of all mice, CUHM008 (mismatching) or CUHM009 (autologous) melanoma tissue was implanted on both flanks and one shoulder of all mice When tumors had reached an average volume of 25 mm3, one arm of each group of mice was treated with 10 mg/kg nivolumab by intraperitoneal injection twice a week while measuring tumor growth. A sematic of the exemplary procedure is provide in FIG. 15A.

Tumors autologous (CHUM009) or allogeneic (CUHM008) to the STOCs were implanted on all HM within a week of the first recorded T cell presence in the periphery, and nivolumab treatment commenced one week subsequent to implantation and continued for the next several weeks. During this period, we observed that nivolumab treatment was ineffective in the control HM, had a more pronounced effect on the growth of allogenic tumors, and significantly reduced that rate at which autologous tumors grew. The final tumor volumes in these mouse groups indicate that nivolumab-treated tumors grew to approximately 50% of the size of untreated tumors. Although both untreated and nivolumab-treated aHM_{T} mice contained small populations of CD8+ T cells in their STOC tissue (as might be expected in an HLA-A matched HM), only the nivolumab-treated animals also had a CD4+ population and a small number CD4/8+ of T cells, indicative of active T cell education. Further, IHC analysis of the STOC tissue validated its thymic architecture and indicated that it expresses human CK5 and CK8, as would be expected of true thymic tissue. Additionally, in aHM_{T} nivolumab-treated animals, the accumulation of T cells within tumor tissues corresponds with a notable increase in Ki67+ tumor cells, implying that their prolonged presence prompts rapid tumor growth, presumably among cells selected against T cell attack.

Combined tumor burden of unmodified humanized mice (FIGS. 15B-15C) or thymectomized STOC009 humanized mice (FIGS. 15D-15E), either with no treatment (green) or treatment with the PD-1 inhibitor nivolumab was measured up to 70 days post-implantation. A Cox Proportional Hazard Analysis assigned a p-value of 0.046 to progressive tumor growth (20% increase) in the aHM_{T} nivolumab-treated arm.

At the end of the experiment, during tissue harvest, the murine thymus (HM control mice) or STOC tissue (mHM_{T} and aHM_{T} mice) were collected, and resident CD3/45+ T cells were identified and quantified by cytometry (Table 12). Cytometric identification of T cells among unmodified HM indicated that few T cells were present in the murine thymus, regardless of nivolumab treatment. Likewise, mHM_{T} had few T cells. When aHM_{T} groups were examined, a large T cell population was observed only after nivolumab treatment.

TABLE 12
Tumor-infiltrating T cells

| | CUHM008 | CUHM008 | CUHM009 | CUHM009 | | | | |
|---|---|---|---|---|---|---|---|---|
| | control, | control, | control, | control, | mHMT, | mHMT, | aHMT, | aHMT, |
| | untreated | nivo | untreated | nivo | untreated | nivo | untreated | nivo |
| Trial 1 | 0 | 0 | 0 | 0 | 0 | 0.01 | 0.003 | 0 |
| Trial 2 | 0.01 | 0.017 | | 0.13 | 0 | 0 | 0.012 | 0 |
| Avg | 0.005 | 0.0085 | 0 | 0.065 | 0 | 0.005 | 0.0075 | 0.026 |
| St. Dev | 0.0070711 | 0.0120208 | 0 | 0.0919239 | 0 | 0.007071068 | 0.006364 | 0.0450333 |
| St. Error | 0.005 | 0.0085 | 0 | 0.065 | 0 | 0.005 | 0.0045 | 0.0318434 |

Cytometric identification of CD45RA+ (naïve) and CD45RO+ (memory) T cells isolated from untreated and nivolumab-treated aHM_{T} spleens was performed (FIG. 16). Nivolumab treatment produced greater populations of both classes of T cells.

At the end of the experiment, tumor tissue was collected from the mice and infiltrating T cells were identified by cytometry. FIG. 17 shows a graph depicting quantification of the flow cytometry results. Analysis of Ki67 expression by live tumor cells was also assessed by IHC (Table 13). Expression after nivolulmab treatment increased only in aHM_{T} tumors.

TABLE 13
Dividing tumor cells after treatment

| | Live | |
|---|---|---|
| | Ki67 + cells | Difference |
| Mouse | (%) | (%) |
| CUHM008 control, untreated | 70.75 | 82.3 |
| CUHM008 control, nivo | 58.22 | |
| CUHM009 control, untreated | 60.92 | 116.6 |
| CUHM009 control, nivo | 71.03 | |
| mHMT, untreated | 74.78 | 95.2 |
| mHMT, nivo | 71.2 | |
| aHMT, untreated | 15.14 | 403.2 |
| aHMT, nivo | 61.04 | |

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as can be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A composition comprising: a plurality of mammalian thymic cells comprising thymic stromal cells and thymic epithelial cells expressing one or more marker comprising EPCAM, CD205 and FOXN1; and a plurality of mammalian hematopoietic cells (HPCs) expressing one or more of CD34+ and CD45+.

2. The composition according to claim 1, wherein the HPCs are derived from a subject to be treated, cord blood, differentiated pluripotent stem cells or other stem cell populations.

3. The composition according to claim 1, further comprising a plurality of mesenchymal cells expressing PDGFRα.

4. A composition comprising: a plurality of mammalian thymic cells comprising thymic stromal cells and thymic epithelial cells expressing one or more marker of EPCAM, CD205 and FOXN1; and a plurality of mesenchymal cells expressing PDGFRα.

5. The composition according to claim 4, further comprising a plurality of HPCs expressing one or more of CD34+ and CD45+.

6. The composition according to claim 3, wherein the mesenchymal cells are derived from mammalian thymi or differentiated HPSCs (human pluripotent stem cells) or other stem cells or splanchnic mesenchyme.

7. The composition according to claim 1, wherein the composition has been cultured for about 1 week to about 3 months.

8. The composition according to claim 7, wherein the mammalian thymic cells express one or more of AIRE, CD205, EPCAM, FOXN1, KRT8, KRT8, DLL1, DLL4, PD-L1, AIRE, HLA-DQ (class II), CD104, PAX9, SIX1, PSMB11, CCL21, CXCL12, RANK, CD80, CD86, Beta 5T, and Tissues restricted antigens (TRAs).

9. The composition according to claim 8, wherein the TRAs comprise one or more of INS, IA2, GAD1, Myelin Basic Protein and Thyroglobulin.

10. The composition according to claim 7, wherein when mammalian HPCs are present, the mammalian HPCs express one or more of CD5, CD7, CD43, CD3, TCRa/b, CD4, CD8, PD1, CD125, NCAM, CD34, VECAD, CD90, CD27, CD38, CD43, CD48, CD117, Sca-1 and CD150.

11. The composition according to claim 7, wherein the mesenchyme when present expresses one or more of CD105, CD90, CD73, VIM, PDGFRβ, FGF7, FGF10, and TE-7.

12. A method for generating functional thymic tissues comprising: obtaining a composition according to claim 1, to comprising a mixed cell culture and incubating the mixed cell culture for about 1 week to about 3 months; and generating functional thymic tissue.

13. The method according to claim 12, wherein the mammalian thymic cells express one or more of AIRE, CD205, EPCAM, FOXN1, KRT8, KRT8, DLL1, DLL4, PD-L1, AIRE, HLA-DQ (class II), CD104, PAX9, SIX1, PSMB11, CCL21, CXCL12, RANK, CD80, CD86, Beta 5T, and Tissues restricted antigens (TRAs).

14. The method according to claim 13, wherein the TRAs comprise one or more of INS, IA2, GAD1, Myelin Basic Protein and Thyroglobulin.

15. The method according to claim 12, wherein at least one of when mammalian HPCs are present, the mammalian HPCs express one or more of CD5, CD7, CD43, CD3, TCRa/b, CD4, CD8, PD1, CD125, NCAM, CD34, VECAD, CD90, CD27, CD38, CD43, CD48, CD117, Sca-1 and CD150; and wherein when present, the mesenchymal cells express one or more of CD105, CD90, CD73, VIM, PDGFRβ, FGF7, FGF10, and TE-7.

16. (canceled)

17. A method for treating a subject having a health condition comprising: at least one of administering a pharmaceutically acceptable population of cells created by the method according to claim 12 to the subject; or administering a pharmaceutically acceptable composition according to claim 7 to the subject.

18. The method for treating a subject according to claim 17, wherein the subject has a at least one of, a condition of the thymus and an immune disorder.

19. (canceled)

20. The method for treating a subject according to claim 19, wherein the immune disorder comprises an adaptive immune disorder or autoimmune disorder.

21. The method for treating a subject according to claim 17, wherein the subject has cancer, an infection or other disorder in need of adaptive immune therapy.

22. A kit comprising a composition according to claim 1, and at least one container.
